SEQUENCE LISTING (35 sequences)

SEQ ID NO: 1 - protein, 76 aa, Artificial
  Synthetic
ADNNFNREQQNAFYEILNMPNLNEEQRNGFIQSLRDDPSQSANLLSEARRLNESQAPGGGGGCADDDDDDHHHHHH

SEQ ID NO: 2 - protein, 88 aa, Artificial
  Synthetic
AYRLILNGRTLRGETTTEAVDAATAERVFRQYANDNGVDGEWTYDDATRTFTVTERPEVIDASELTPAVTGGGGCADDDDDDHHHHHH

SEQ ID NO: 3 - protein, 79 aa, Artificial
  Synthetic
ATIRANLIYADGRTQTAEFRGTFEEATAEAYRYADLLARENGRYTVDVADRGYTLNIRFAGGGGGCADDDDDDHHHHHH

SEQ ID NO: 4 - protein, 62 aa, Artificial
  Synthetic
ADNNFNREQQNAFYEILNMPNLNEEQRNGFIQSLRDDPSQSANLLSEARRLNESQAPGGGGG

SEQ ID NO: 5 - protein, 74 aa, Artificial
  Synthetic
AYRLILNGRTLRGETTTEAVDAATAERVFRQYANDNGVDGEWTYDDATRTFTVTERPEVIDASELTPAVTGGGG

SEQ ID NO: 6 - protein, 65 aa, Artificial
  Synthetic
ATIRANLIYADGRTQTAEFRGTFEEATAEAYRYADLLARENGRYTVDVADRGYTLNIRFAGGGGG

SEQ ID NO: 7 - protein, 58 aa, Staphylococcus sp.
ADNNFNKEQQNAFYEILNMPNLNEEQRNGFIQSLKDDPSQSANLLSEAKKLNESQAPK

SEQ ID NO: 8 - protein, 70 aa, Streptococcus sp.
TYKLILNGKTLKGETTTEAVDAATAEKVFKQYANDNGVDGEWTYDDATKTFTVTERPEVIDASELTPAVT

SEQ ID NO: 9 - protein, 60 aa, Peptostreptococcus sp.
VTIKANLIYADGKTQTAEFKGTFEEATAEAYRYADLLAKENGKYTVDVADKGYTLNIKFA

SEQ ID NO: 10 - protein, 76 aa, Artificial
  Synthetic
MADNNFNKEQQNAFYEILNMPNLNEEQRNGFIQSLKDDPSQSANLLSEAKKLNESQAPGGGGGCADDDDDHHHHHH

SEQ ID NO: 11 - DNA, 321 bp, Artificial
  Synthetic
ggatccttgacaatatcttaactatctgttataatatattgaccaggttaactaactaagcagcaaaaggaggaacgactatggctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaaggtggcggtggctgcgctgatgacgatgacgatgaccatcatcaccaccatcattaagaattcc

SEQ ID NO: 12 - protein, 89 aa, Artificial
  Synthetic
MAYRLILNGRTLRGETTTEAVDAATAERVFRQYANDNGVDGEWTYDDATRTFTVTERPEVIDASELTPAVTGGGGCADDDDDDHHHHHH

SEQ ID NO: 13 - DNA, 355 bp, Artificial
  Synthetic
ggatccttgacaatatcttaactatctgttataatatattgaccaggttaactactaagcagcaaaaggaggaacgactatggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactggtggcggtggctgcgctgatgacgatgacgatgaccatcatcaccaccatcattaagaattc

SEQ ID NO: 14 - protein, 80 aa, Artificial
  Synthetic
MATIRANLIYADGRTQTAEFRGTFEEATAEAYRYADLLARENGRYTVDVADRGYTLNIRFAGGGGGCADDDDDDHHHHHH

SEQ ID NO: 15 - DNA, 329 bp, Artificial
  Synthetic
ggatccttgacaatatcttaactatctgttataatatattgaccaggttaactaactaagcagcaaaaggaggaacgactatggctactattcgtgctaatctgatttatgctgatggtcgtactcagactgctgagtttcgtggtacttttgaggaggctactgctgaggcttatcgttatgctgatctgctggctcgtgagaatggtcgttatactgttgatgttgctgatcgtggttatactctgaatattcgttttgctggtggtggcggtggctgcgctgatgacgatgacgatgaccatcatcaccaccatcattaagaattc

SEQ ID NO: 16 - protein, 4 aa, Artificial
  Synthetic
GGGG

SEQ ID NO: 17 - protein, 6 aa, Artificial
  Synthetic
HQHQHQ

SEQ ID NO: 18 - protein, 10 aa, Artificial
  Synthetic
EQKLISEEDL

SEQ ID NO: 19 - protein, 9 aa, Artificial
  Synthetic
YPYDVPDYA

SEQ ID NO: 20 - protein, 8 aa, Artificial
  Synthetic
DYKDDDDK

SEQ ID NO: 21 - protein, 6 aa, Artificial
  Synthetic
DDDDDD

SEQ ID NO: 22 - protein, 6 aa, Artificial
  Synthetic
HHHHHH

SEQ ID NO: 23 - protein, 5 aa, Artificial
  Synthetic
SGGGG

SEQ ID NO: 24 - protein, 58 aa, Staphylococcus sp.
ADNNFNREQQNAFYEILNMPNLNEEQRNGFIQSLRDDPSQSANLLSEARRLNESQAPG

SEQ ID NO: 25 - protein, 72 aa, Streptococcus sp.
AYRLILNGRTLRGETTTEAVDAATAERVFRQYANDNGVDGEWTYDDATRTFTVTERPEVIDASELTPAVTPG

SEQ ID NO: 26 - protein, 62 aa, Peptostreptococcus sp.
ATIRANLIYADGRTQTAEFRGTFEEATAEAYRYADLLARENGRYTVDVADRGYTLNIRFAPG

SEQ ID NO: 27 - DNA, 509 bp, Artificial
  Synthetic
ggatccttgacaatatcttaactatctgttataatatattgaccaggttaactaactaagcagcaaaaggaggaacgactatgtcgggcggtggtggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagccccgggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagcaccgggtggtggcggtggctgcgctgatgacgatgacgatgaccatcatcaccaccatcattaagaattc

SEQ ID NO: 28 - DNA, 180 bp, Artificial
  Synthetic
cccgggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagccccggg

SEQ ID NO: 29 - DNA, 683 bp, Artificial
  Synthetic
ggatccttgacaatatcttaactatctgttataatatattgaccaggttaactaactaagcagcaaaaggaggaacgactatgtcgggcggtggtggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagccccgggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagccccgggtgctgataacaatttcaaccgtgaacaacaaaatgctttctatgaaatcttgaatatgcctaacttaaacgaagaacaacgcaatggtttcatccaaagcttacgtgatgacccaagccaaagtgctaacctattgtcagaagctcgtcgtttaaatgaatctcaagcaccgggtggtggcggtggctgcgctgatgacgatgacgatgaccatcatcaccaccatcattaagaattc

SEQ ID NO: 30 - DNA, 572 bp, Artificial
  Synthetic
ggatccttgacaatatcttaactatctgttataatatattgaccaggttaactaactaagcagcaaaaggaggaacgactatggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactcccggggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactggtggcggtggctgcgctgatgacgatgacgatgaccatcatcaccaccatcattaagaattc

SEQ ID NO: 31 - DNA, 222 bp, Artificial
  Synthetic
cccggggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactcccggg

SEQ ID NO: 32 - DNA, 788 bp, Artificial
  Synthetic
ggatccttgacaatatcttaactatctgttataatatattgaccaggttaactaactaagcagcaaaaggaggaacgactatggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactcccggggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactcccggggcttaccgtttaatccttaatggtcgtacattgcgtggcgaaacaactactgaagctgttgatgctgctactgcagaacgtgtcttccgtcaatacgctaacgacaacggtgttgacggtgaatggacttacgacgatgcgactcgtacctttacggtaactgaacgtcctgaggttattgatgcttcggagctgactcctgctgttactggtggcggtggctgcgctgatgacgatgacgatgaccatcatcaccaccatcattaagaattc

SEQ ID NO: 33 - DNA, 515 bp, Artificial
  Synthetic
ggatccttgacaatatcttaactatctgttataatatattgaccaggttaactaactaagcagcaaaaggaggaacgactatggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctcccggggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctggtggtggcggtggctgcgctgatgacgatgacgatgaccatcatcaccaccatcattaagaattc

SEQ ID NO: 34 - DNA, 193 bp, Artificial
  Synthetic
cccggggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctcccgggg

SEQ ID NO: 35 - DNA, 701 bp, Artificial
  Synthetic
ggatccttgacaatatcttaactatctgttataatatattgaccaggttaactaactaagcagcaaaaggaggaacgactatggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctcccggggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctcccggggctactattcgtgctaatctgatttatgctgatggtcgtactcagactgctgagtttcgtggtacttttgaggaggctactgctgaggcttatcgttatgctgatctgctgcctcgtgagaatggtcgttatactgttgatgttgctgatcgtggttatactctgaatattcgttttgctggtggtggcggtggctgcgctgatgacgatgacgatgaccatcatcaccaccatcattaagaattc

(19) United States
(12) Patent Application Publication
(10) Pub. No.: US 2009/0299035 A1
(43) Pub. Date: Dec. 3, 2009

(54) PROTEIN APPROPRIATE FOR ORIENTATION-CONTROLLED IMMOBILIZATION AND IMMOBILIZATION CARRIER ON WHICH THE PROTEINS ARE IMMOBILIZED

(75) Inventors: Masahiro Iwakura, Ibaraki (JP); Kiyonori Hirota, Ibaraki (JP); Hiroyuki Sota, Ibaraki (JP); Gou Sarara, Ibaraki (JP); Yukiko Aruga, Ibaraki (JP); Chiori Yamane, Ibaraki (JP)

Correspondence Address: FOLEY AND LARDNER LLP;SUITE 500, 3000 K STREET NW, WASHINGTON, DC, 20007, US

(73) Assignee: National Institute of Advanced Idustrial Science and Technology

(21) Appl. No.: 12/443,623

(22) PCT Filed: Oct. 10, 2007
(86) PCT No.: PCT/JP2007/069722

§ 371 Date: Mar. 30, 2009

(30) Foreign Application Priority Data
Oct. 10, 2006 (JP) 2006-276468
Mar. 7, 2007 (JP) 2007-057791

(51) Int. Cl. C07K 14/00

(52) U.S. Cl. 530/324

(57) ABSTRACT

An object of the present invention is to provide a novel protein having the following amino acid sequence altered for specifically and efficiently binding a protein to an immobilization carrier via the carboxy terminus. The protein is used for immobilizing a portion represented by R1-R2 on an immobilization carrier, comprising the amino acid sequence represented by the general formula R1-R2-R3-R4-R5 [wherein:
the sequences are oriented from the amino terminal side to the carboxy terminal side;
the sequence of the R1 portion is the sequence of a subject protein to be immobilized and contains neither a lysine residue nor a cysteine residue;
the sequence of the R2 portion may be absent, but when the sequence of the R2 portion is present, the sequence of the R2 portion is a spacer sequence composed of amino acid residues other than lysine and cysteine residues;
the sequence of the R3 portion is composed of two residues of amino acid represented by cysteine-X (where X denotes an amino acid residue other than lysine or cysteine);
the sequence of the R4 portion may be absent, but when the sequence of the R4 portion is present, the sequence of the R4 portion contains neither a lysine residue nor a cysteine residue, but contains an acidic amino acid residue capable of acidifying the isoelectric point of the entire protein comprising the amino acid sequence represented by the general formula R1-R2-R3-R4-R5; and
the sequence of an R5 portion is an affinity tag sequence for protein purification.

### TECHNICAL FIELD

The present invention relates to an immobilized protein. The present invention further relates to an immobilization carrier on which the proteins are immobilized in an orientation-controlled manner and a method for immobilizing the protein.

### BACKGROUND ART

It has been attempted to use a soluble protein as an immobilization protein by binding it to, for example, an insoluble immobilization carrier such as agarose gel. Examples of such attempts are the development of an immobilized enzyme prepared by binding an enzyme protein to an immobilization carrier and the production of an enzyme reactor utilizing the same. It is desired that such immobilized proteins have qualities such as: uniform properties and functions; retention of properties and functions equivalent to those of unimmobilized soluble proteins; and the ability to allow a higher amount of an immobilized protein per carrier. These qualities depend on methods for protein immobilization.

A protein immobilization method mainly comprises chemically binding a protein to an immobilization carrier using the reactivity of a side chain of an amino acid composing the protein. However, as long as such an immobilization reaction using the functional groups of side chains is employed, specifically, when a protein has a plurality of side chains to be used for an immobilization reaction, it is difficult to control immobilization sites, to prevent immobilization from occurring at a plurality of positions, and to maintain the homogeneity of immobilized proteins. Factors relating to such difficulties can lead to hypofunctions of immobilized proteins. Thus, improvement has been desired.

To avoid heterogeneity of immobilized proteins due to immobilization via functional groups of a plurality of side chains, it has been attempted to design and prepare a protein sequence having a sole functional group by subjecting a protein to amino acid substitution or the like. An example of such attempt that has been performed involves altering a sequence such that it has only one cysteine residue in a protein and then carrying out site-specific immobilization via an S—S bond or the like (see JP Patent No. 2517861; M. Iwakura et al. (1993) J. Biochem. 114, 339-343; S. J. Vigmond et al.(1994) Langumur, 10, 2860-2862; and M. Iwakura et al. (1995) J. Biochem. 117, 480-488).

Meanwhile, only one carboxy terminus is present in a protein. Hence, carboxy terminus-mediated immobilization is carried out, so that site-specific and orientation-controlled immobilization can be carried out. The present inventors have previously developed a method utilizing a cyanocysteine-residue-mediated amide bond forming reaction, by which a carboxyl group at the carboxy terminus of a protein is immobilized on a carrier having a primary amine via a peptide (amide) bond (see JP Patent Nos. 3788828, 2990271, and 3047020 and JP Patent Publication (Kokai) No. 2003-344396 A). Accordingly, each immobilized protein is bound at one position (the carboxy terminus) via the main chain, so that the thus obtained proteins are immobilized in an orientation-controlled manner and are completely homogenous. Furthermore, orientation control and homogeneity are maintained, so that the reversibility of denaturation of immobilized proteins can be enhanced and properties that are excellent in terms of usefulness can be added, such that heat sterilization of immobilized proteins is made possible (see M. Iwakura et al. (2001) Protein engineer., 14, 583-589).

As described above, the immobilization technique utilizing a cyanocysteine-mediated binding reaction that has been developed by the present inventors has good characteristics, but is problematic in that: the production of proteins to be immobilized may be difficult depending on proteins to be used; proteins should be treated differently according to the properties of the proteins; and an insoluble immobilization carrier is required to contain a large amount of a primary amine as a functional group. Hence, the development of a technique for removing ion interactions or the like resulting from the reactivity or the like of unreacted primary amines remaining on immobilization carriers after an immobilization reaction has remained as an object to be achieved, for example.

### DISCLOSURE OF THE INVENTION

### Objects to be Achieved by the Invention

An object of the present invention is to reveal and specify conditions under which the amino acid sequence of a protein that contains the amino acid sequence of a specific protein to be immobilized is optimized for cyanocysteine-mediated orientation-controlled immobilization.

### Means to Achieve the Object

The present inventors have conducted intensive studies to solve the above problems in protein immobilization. Specifically, the present inventors have conducted intensive studies to convert the amino acid sequence of an immobilization protein that contains the amino acid sequence of a subject protein to be immobilized to a sequence appropriate for cyanocysteine-mediated orientation-controlled immobilization. Thus, the present inventors have discovered that the above objects can be achieved by designing a sequence comprising 5 portions (that include a portion comprising the amino acid sequence of a subject protein to be immobilized); that is, the sequence represented by R1-R2-R3-R4-R5 and then causing each portion to have characteristics. Furthermore, the present inventors have also revealed that separation and purification after preparation of a gene corresponding to an immobilization protein and the following expression in host cells can also be standardized; and that immobilization reaction conditions can also be standardized. Furthermore, the present inventors have also discovered that a plurality of functions such as binding ability, which are exerted separately by individual repeating sequence portions, can be imparted to a single polypeptide chain by, in the above sequence represented by R1-R2-R3-R4-R5, preparing the sequence of R1 comprising two portions represented by P-Q, the sequence of the P portion comprising (Ser or Ala)-(Gly) n (where n is any one integer ranging from 1 to 10), and the protein sequence of the Q portion having a repeating unit, in which the sequence unit containing neither a lysine residue nor a cysteine residue is repeated. The present inventors have further discovered that this can enhance the functions. Thus, the present inventors have completed the present invention.

The embodiments of the present invention are as follows.

[1] A protein to be used for immobilizing a portion of the protein represented by R1-R2 on an immobilization carrier, comprising an amino acid sequence represented by the general formula R1-R2-R3-R4-R5, wherein:

the sequences are oriented from the amino terminal side to the carboxy terminal side,

the sequence of the R1 portion is the sequence of a subject protein to be immobilized and contains neither a lysine residue nor a cysteine residue;

the sequence of the R2 portion may be absent, but when the sequence of the R2 portion is present, the sequence of the R2 portion is a spacer sequence composed of amino acid residues other than lysine and cysteine residues;

the sequence of the R3 portion is composed of two residues of amino acid represented by cysteine-X (where X denotes an amino acid residue other than lysine or cysteine);

the sequence of the R4 portion may be absent, but when the sequence of the R4 portion is present, the sequence of the R4 portion contains neither a lysine residue nor a cysteine residue, but contains an acidic amino acid residue capable of acidifying the isoelectric point of the entire protein comprising the amino acid sequence represented by the general formula R1-R2-R3-R4-R5; and

the sequence of an R5 portion is an affinity tag sequence for protein purification.

[2] The protein according to [1] comprising the amino acid sequence represented by the general formula R1-R2-R3-R4-R5, wherein, in the amino acid sequence of the general formula R1-R2-R3-R4-R5, the sequence of the R1 portion is: the amino acid sequence of a naturally derived protein; or
the amino acid sequence of a protein that comprises an amino acid sequence altered to contain neither a lysine residue nor a cysteine residue and has functions equivalent to those of the naturally derived protein, in which the altered amino acid sequence is obtained by substituting all lysine and cysteine residues in the amino acid sequence of the naturally derived protein with amino acid residues other than lysine and cysteine residues.
[3] The protein according to [1] comprising the amino acid sequence represented by the general formula R1-R2-R3-R4-R5, wherein, in the amino acid sequence of the general formula R1-R2-R3-R4-R5, the sequence of the R2 portion comprises 1 to 10 glycines.
[4] The protein according to [1] comprising the amino acid sequence represented by the general formula R1-R2-R3-R4-R5, wherein, in the amino acid sequence of the general formula R1-R2-R3-R4-R5, the sequence of the R4 portion comprises 1 to 10 amino acid residues of aspartic acid and/or glutamic acid.
[5] The protein according to [1] comprising the amino acid sequence represented by the general formula R1-R2-R3-R4-R5, wherein, in the amino acid sequence of the general formula R1-R2-R3-R4-R5, the sequence of the R5 portion is an amino acid sequence comprising 4 or more histidine residues.
[6] The protein according to any one [1] to [5], wherein, in the amino acid sequence of the general formula R1-R2-R3-R4-R5, the sequence of the R1 portion has a function of interacting specifically with an antibody molecule.
[7] The protein according to [1], comprising the following amino acid sequence (SEQ ID NO: 1):

Ala-Asp-Asn-Asn-Phe-Asn-Arg-Glu-Gln-Gln
Asn-Ala-Phe-Tyr-Glu-Ile-Leu-Asn-Met-Pro
Asn-Leu-Asn-Glu-Glu-Gln-Arg-Asn-Gly-Phe
Ile-Gln-Ser-Leu-Arg-Asp-Asp-Pro-Ser-Gln
Ser-Ala-Asn-Leu-Leu-Ser-Glu-Ala-Arg-Arg
Leu-Asn-Glu-Ser-Gln-Ala-Pro-Gly-Gly-Gly
Gly-Gly-Cys-Ala-Asp-Asp-Asp-Asp-Asp-Asp
His-His-His-His-His-His

[8] The protein according to [1], comprising the following sequence (SEQ ID NO: 2):

Ala-Tyr-Arg-Leu-Ile-Leu-Asn-Gly-Arg-Thr
Leu-Arg-Gly-Glu-Thr-Thr-Thr-Glu-Ala-Val
Asp-Ala-Ala-Thr-Ala-Glu-Arg-Val-Phe-Arg
Gln-Tyr-Ala-Asn-Asp-Asn-Gly-Val-Asp-Gly
Glu-Trp-Thr-Tyr-Asp-Asp-Ala-Thr-Arg-Thr
Phe-Thr-Val-Thr-Glu-Arg-Pro-Glu-Val-Ile
Asp-Ala-Ser-Glu-Leu-Thr-Pro-Ala-Val-Thr
Gly-Gly-Gly-Gly-Cys-Ala-Asp-Asp-Asp-Asp
Asp-Asp-His-His-His-His-His-His
Ala-Thr-Ile-Arg-Ala-Asn-Leu-Ile-Tyr-Ala
Asp-Gly-Arg-Thr-Gln-Thr-Ala-Glu-Phe-Arg
Gly-Thr-Phe-Glu-Glu-Ala-Thr-Ala-Glu-Ala

[9] The protein according to [1], comprising the following sequence (SEQ ID NO: 3):

Ala-Thr-Ile-Arg-Ala-Asn-Leu-Ile-Tyr-Ala
Asp-Gly-Arg-Thr-Gln-Thr-Ala-Glu-Phe-Arg
Gly-Thr-Phe-Glu-Glu-Ala-Thr-Ala-Glu-Ala
Tyr-Arg-Tyr-Ala-Asp-Leu-Leu-Ala-Arg-Glu
Asn-Gly-Arg-Tyr-Thr-Val-Asp-Val-Ala-Asp
Arg-Gly-Tyr-Thr-Leu-Asn-Ile-Arg-Phe-Ala
Gly-Gly-Gly-Gly-Gly-Cys-Ala-Asp-Asp-Asp
Asp-Asp-Asp-His-His-His-His-His-His

[10] An immobilization carrier, to which a protein comprising the amino acid sequence represented by the general formula R1-R2-R3-R4-R5 according to any one of [1] to [9] is adsorbed via electrostatic interactions.
[11] A method for preparing an immobilized protein, comprising converting a sulfhydryl group of the sole cysteine residue existing in the protein according to any one of [1] to [9] to a thiocyano group, causing the resultant to act on an immobilization carrier having a primary amine as a functional group, and then binding an amino acid sequence portion existing on the amino terminal side from the cysteine residue in the protein to the immobilization carrier via an amide bond.
[12] A carrier on which a protein is immobilized, wherein a sulfhydryl group of the sole cysteine residue existing in the protein according to any one of [1] to [9] is converted to a thiocyano group and then the resultant is caused to act on an arbitrary immobilization carrier having a primary amine as a functional group, so as to bind an amino acid sequence portion existing on the amino terminal side from the cysteine residue in the protein via an amide bond.
[13] The immobilization carrier on which a protein is immobilized according to [12], wherein the carboxy terminus of a protein comprising the following sequence (SEQ ID NO: 4) binds to an immobilization carrier having a primary amine as a functional group via an amide bond:

Ala-Asp-Asn-Asn-Phe-Asn-Arg-Glu-Gln-Gln-Asn-Ala-
Phe-Tyr-Glu-Ile-Leu-Asn-Met-Pro-Asn-Leu-Asn-Glu-
Glu-Gln-Arg-Asn-Gly-Phe-Ile-Gln-Ser-Leu-Arg-Asp-
Asp-Pro-Ser-Gln-Ser-Ala-Asn-Leu-Leu-Ser-Glu-Ala-
Arg-Arg-Leu-Asn-Glu-Ser-Gln-Ala-Pro-Gly-Gly-Gly-
Gly-Gly.

[14] The immobilization carrier on which a protein is immobilized according to [12], wherein the carboxy terminus of a protein comprising the following sequence (SEQ ID NO: 5) binds to an immobilization carrier having a primary amine as a functional group via an amide bond:

Ala-Tyr-Arg-Leu-Ile-Leu-Asn-Gly-Arg-Thr-Leu-Arg-
Gly-Glu-Thr-Thr-Thr-Glu-Ala-Val-Asp-Ala-Ala-Thr-
Ala-Glu-Arg-Val-Phe-Arg-Gln-Tyr-Ala-Asn-Asp-Asn-
Gly-Val-Asp-Gly-Glu-Trp-Thr-Tyr-Asp-Asp-Ala-Thr-
Arg-Thr-Phe-Thr-Val-Thr-Glu-Arg-Pro-Glu-Val-Ile-
Asp-Ala-Ser-Glu-Leu-Thr-Pro-Ala-Val-Thr-Gly-Gly-
Gly-Gly.

[15] The immobilization carrier on which a protein is immobilized according to [12], wherein the carboxy terminus of a protein represented by the following sequence (SEQ ID NO: 6) binds to an immobilization carrier having a primary amine as a functional group via an amide bond:

Ala-Thr-Ile-Arg-Ala-Asn-Leu-Ile-Tyr-Ala-Asp-Gly-
Arg-Thr-Gln-Thr-Ala-Glu-Phe-Arg-Gly-Thr-Phe-Glu-
Glu-Ala-Thr-Ala-Glu-Ala-Tyr-Arg-Tyr-Ala-Asp-Leu-
Leu-Ala-Arg-Glu-Asn-Gly-Arg-Tyr-Thr-Val-Asp-Val-
Ala-Asp-Arg-Gly-Tyr-Thr-Leu-Asn-Ile-Arg-Phe-Ala-
Gly-Gly-Gly-Gly-Gly

[16] A method for designing a protein comprising the amino acid sequence represented by the general formula R1-R2-R3-R4-R5 to be used for immobilizing a protein comprising the amino acid sequence represented by R1-R2 on an immobilization carrier, so that the amino acid sequences of the R1, R2, R3, R4, and R5 portions are selected to meet the following conditions:
(a) the sequence of the R1 portion is the sequence of a protein to be immobilized containing neither a lysine residue nor a cysteine residue;
(b) the sequence of the R2 portion is absent or the sequence of the R2 portion is a spacer sequence composed of amino acid residues other than lysine and cysteine residues when the sequence of the R2 portion is present;
(c) the sequence of the R3 portion is a sequence composed of two residues of amino acid represented by cysteine-X (where X denotes an amino acid residue other than lysine or cysteine);
(d) the sequence of the R4 portion is absent or the sequence of the R4 portion is a sequence containing neither a lysine residue nor a cysteine residue, but containing an acidic amino acid residue capable of acidifying the isoelectric point of the entire protein comprising the amino acid sequence represented by the general formula R1-R2-R3-R4-R5 when the sequence of the R4 portion is present; and
(e) the sequence of the R5 portion is an affinity tag sequence for protein purification.
[17] The protein according to [1] to be used for immobilizing the portion represented by R1-R2 on an immobilization carrier, comprising the amino acid sequence represented by the general formula R1-R2-R3-R4-R5, wherein the sequence of the R1 portion is represented by P-Q, the sequence of the P portion may be absent or present and is a sequence comprising (Ser or Ala)-(Gly)n (where n denotes an arbitrary integer ranging from 1 to 10) when present, and the sequence of the Q portion is the sequence of a protein having a repeating unit in which a sequence unit containing neither a lysine residue nor a cysteine residue is repeated.
[18] The protein according to [17] comprising the amino acid sequence represented by the general formula R1-R2-R3-R4-R5, wherein, in the amino acid sequence represented by P-Q, the sequence of the repeating unit of the Q portion is the amino acid sequence of a naturally derived protein or the amino acid sequence of a protein that comprises an amino acid sequence altered to contain neither a lysine residue nor a cysteine residue, which is obtained by substituting all lysine and cysteine residues in the amino acid sequence of the naturally derived protein with amino acid residues other than lysine and cysteine residues and has functions equivalent to those of the naturally derived protein.
[19] The protein according to [17] comprising the amino acid sequence represented by the general formula R1-R2-R3-R4-R5, wherein, in the amino acid sequence represented by the general formula R1-R2-R3-R4-R5, the sequence of the R2 portion comprises 1 to 10 glycines.
[20] The protein according to [17] comprising the amino acid sequence represented by the general formula R1-R2-R3-R4-R5, wherein, in the amino acid sequence represented by the general formula R1-R2-R3-R4-R5, the sequence of the R4 portion comprises 2 to 10 amino acid residues comprising 2 types of amino acid residue, aspartic acid and glutamic acid.
[21] The protein according to [17] comprising the amino acid sequence represented by the general formula R1-R2-R3-R4-R5, wherein, in the amino acid sequence represented by the general formula R1-R2-R3-R4-R5, the sequence of the R5 portion is an amino acid sequence comprising 4 or more histidine residues.
[22] The protein according to any one of [17] to [21], wherein, in the amino acid sequence represented by P-Q, the sequence of the repeating unit of the Q portion has a function of interacting specifically with an antibody molecule.

[23] The protein according to [17], wherein, in the amino acid sequence represented by the general formula R1-R2-R3-R4-R5, the sequence of the R1 portion is represented by P-Q,

### P=Ser-Gly-Gly-Gly-Gly,

Q=(Ala-Asp-Asn-Asn-Phe-Asn-Arg-Glu-Gln-Gln-Asn-Ala-Phe-Tyr-Glu-Ile-Leu-Asn-Met-Pro-Asn-Leu-Asn-Glu-Glu-Gln-Arg-Asn-Gly-Phe-Ile-Gln-Ser-Leu-Arg-Asp-Asp-Pro-Ser-Gln-Ser-Ala-Asn-Leu-Leu-Ser-Glu-Ala-Arg-Arg-Leu-Asn-Glu-Ser-Gln-Ala-Pro-Gly) n (where n denotes an arbitrary integer ranging from 2 to 5),

### R2=Gly-Gly-Gly-Gly,

### R3=Cys-Ala,

### R4=Asp-Asp-Asp-Asp-Asp-Asp, and

### R5=His-His-His-His-His-His.

[24] The protein according to [17], wherein, in the amino acid sequence represented by the general formula R1-R2-R3-R4-R5, the sequence of the R1 portion is represented by P-Q,

P=absent,
Q=(Ala-Tyr-Arg-Leu-Ile-Leu-Asn-Gly-Arg-Thr-Leu-Arg-Gly-Glu-Thr-Thr-Thr-Glu-Ala-Val-Asp-Ala-Ala-Thr-Ala-Glu-Arg-Val-Phe-Arg-Gln-Tyr-Ala-Asn-Asp-Asn-Gly-Val-Asp-Gly-Glu-Trp-Thr-Tyr-Asp-Asp-Ala-Thr-Arg-Thr-Phe-Thr-Val-Thr-Glu-Arg-Pro-Glu-Val-Ile-Asp-Ala-Ser-Glu-Leu-Thr-Pro-Ala-Val-Thr-Pro-Gly) n (where n denotes an arbitrary integer ranging from 2 to 5),

### R2=Gly-Gly-Gly-Gly,

### R3=Cys-Ala,

### R4=Asp-Asp-Asp-Asp-Asp-Asp, and

### R5=His-His-His-His-His-His.

[25] The protein according to [17], wherein, in the amino acid sequence represented by the general formula R1-R2-R3-R4-R5, the sequence of the R1 portion is represented by P-Q,
P=absent,
Q=(Ala-Thr-Ile-Arg-Ala-Asn-Leu-Ile-Tyr-Ala Asp-Gly-Arg-Thr-Gln-Thr-Ala-Glu-Phe-Arg Gly-Thr-Phe-Glu-Glu-Ala-Thr-Ala-Glu-Ala Tyr-Arg-Tyr-Ala-Asp-Leu-Leu-Ala-Arg-Glu Asn-Gly-Arg-Tyr-Thr-Val-Asp-Val-Ala-Asp Arg-Gly-Tyr-Thr-Leu-Asn-Ile-Arg-Phe-Ala Pro-Gly-) n (where n denotes an arbitrary integer ranging from 2 to 5),

### R2=Gly-Gly-Gly-Gly,

### R3=Cys-Ala,

### R4=Asp-Asp-Asp-Asp-Asp-Asp, and

### R5=His-His-His-His-His-His.

[26] An immobilization carrier, to which the protein according to any one of [17] to [22] comprising the amino acid sequence represented by the general formula R1-R2-R3-R4-R5 is adsorbed via electrostatic interactions.
[27] A method for preparing an immobilized protein, comprising converting a sulfhydryl group of the sole cysteine residue existing in the protein according to any one of [17] to [22] to a thiocyano group, causing the resultant to act on an immobilization carrier having a primary amine as a functional group, and then binding an amino acid sequence portion existing on the amino terminal side from the cysteine residue in the protein to the immobilization carrier via an amide bond.
[28] An immobilization carrier on which a protein is immobilized, wherein a sulfhydryl group of the sole cysteine residue existing in the protein according to any one of [17] to [22] is converted to a thiocyano group and then the resultant is caused to act on an arbitrary immobilization carrier having a primary amine as a functional group, so as to bind an amino acid sequence portion existing on the amino terminal side from the cysteine residue in the protein to the immobilization carrier via an amide bond.

In addition, naturally derived proteins are composed of 20 types of amino acid residue including cysteine and lysine. It has been unknown whether or not a sequence containing neither cysteine nor lysine as constituent amino acid residues retains biological functions, such as functions for specifically carrying out protein-to-protein recognition and protein-to-protein binding, functions for specifically carrying out protein-to-nucleic acid recognition and protein-to-nucleic acid binding, and catalytic functions, for example. According to the present invention, it has been revealed that a protein altered to contain neither cysteine nor lysine can have functions equivalent to those of its original natural protein.

### EFFECT OF THE INVENTION

According to the present invention, a protein immobilized in an orientation-controlled manner can be prepared efficiently and rapidly by designing an amino acid sequence represented by the general formula R1-R2-R3-R4-R5, preparing a protein comprising the amino acid sequence, and then using the protein for immobilization. Selection is made to satisfy the conditions for each of the R1, R2, R3, R4, and R5 portions, so that all proteins can be immobilized while controlling the orientation. Moreover, a common sequence is used as R5 to be used for purification of the thus designed and prepared protein. Hence, any protein for immobilization can be purified with a common technique regardless of the sequence of R1, which is a subject protein to be immobilized. Furthermore, reaction conditions for immobilization can also be standardized.

Furthermore, in the above sequence represented by R1-R2-R3-R4-R5, R1 is a sequence comprising two portions represented by P-Q, the P portion may be present or absent, but when the P portion is present, the sequence comprises (Ser or Ala)-(Gly) n (where n is an integer between 1 and 10), and the sequence of the Q portion is the sequence of a protein having a repeating unit. The Q portion comprises such sequence in which a sequence unit containing neither a lysine residue nor a cysteine residue is repeated, so that a single polypeptide chain can exert a plurality of functions (that are exerted by each sequence unit), so that an effect of enhancing the functions can be obtained.

### PREFERRED EMBODIMENTS OF THE INVENTION

The present invention will be described in detail as follows.

The term “protein for immobilization comprising an amino acid sequence containing the amino acid sequence of a subject protein to be immobilized” appropriate for orientation-controlled immobilization of the protein of the present invention refers to a protein that is expressed as a protein comprising the amino acid sequence represented by the general formula R1-R2-R3-R4-R5. In such general formula, the sequence is an amino acid sequence oriented from the amino terminal side to the carboxy terminal side. The sequence of the R1 portion is the amino acid sequence of an arbitrary protein to be subjected to immobilization and is characterized by containing neither a lysine residue nor a cysteine residue. The sequence of the R2 portion is an arbitrary spacer sequence composed of amino acid residues other than lysine and cysteine residues. The R2 portion may be absent. The sequence of the R3 portion is composed of 2 residues of amino acid represented by cysteine-X (where X denotes an amino acid residue other than lysine or cysteine). The sequence of the R4 portion is an arbitrary sequence containing neither a lysine residue nor a cysteine residue and is characterized by containing an acidic amino acid residue(s) capable of acidifying the isoelectric point of the entire sequence of R1-R2-R3-R4-R5. The R4 portion may be absent. The sequence of the R5 portion is an arbitrary affinity tag sequence that can bind to a specific compound and is characterized by containing 4 or more histidine residues, for example.

In the protein of the present invention comprising the amino acid sequence represented by the general formula R1-R2-R3-R4-R5, the sequence of the R1 portion is the amino acid sequence of a subject protein to be immobilized and is characterized by containing neither a lysine residue nor a cysteine residue. The number of amino acids in the R1 portion is not limited, so that an amino acid sequence with any number of amino acids can be selected herein according to purposes. The sequence of the R1 portion is a partial amino acid sequence of the amino acid sequence of the subject protein to be immobilized. A protein fragment comprising the amino acid sequence may be a partial amino acid sequence having functions and activity equivalent to those of the above protein. In this case, the sequence of R1 is the amino acid sequence of a functional domain having the functions of a subject protein to be immobilized, for example.

In the case of the present invention, the R1 portion is responsible for target functions. Also, only the R3 portion requires a cysteine residue for immobilization reaction and a primary amine is used as a functional group in a carrier. Therefore, a lysine residue having a cysteine residue and a primary amine group in its side chain is inappropriate as an amino acid residue composing the R1 portion.

Furthermore, in the sequence represented by R1-R2-R3-R4-R5, R1 can be a sequence comprising 2 portions represented by P-Q. In this case, the sequence of the P portion is represented by (Ser or Ala)-(Gly) n (where n denotes an arbitrary integer ranging from 1 to 10) and the sequence of the Q portion is the sequence of a protein having a repeating unit, in which the sequence unit containing neither a lysine residue nor a cysteine residue is repeated. The number of repetition is not limited and preferably ranges from 2 to 5.

Naturally derived proteins are generally composed of 20 types of amino acid residue including lysine and cysteine residues. When the R1 portion that is responsible for target functions contains a lysine residue or a cysteine residue, the residue should be substituted with any one of 18 types of amino acid other than lysine or cysteine such that the resultant can retain the functions of the original natural protein.

The present inventors have already established methods for preparing proteins containing neither cysteine nor methionine (JP Patent Republication No. 01/000797, M. Iwakura et al. J. Biol. Chem. 281, 13234-13246 (2006), JP Patent Publication (Kokai) No. 2005-058059 A). With the use of a method similar to these methods, a protein comprising an amino acid sequence composed of 18 types of amino acid containing neither a cysteine residue nor a lysine residue and exerting functions equivalent to those of a natural protein can be prepared by amino acid sequence conversion based on the amino acid sequence of the naturally derived protein. The outline of this method is as described below.

1. All cysteine residue portions and lysine residue portions in a natural sequence are subjected to extensive single amino acid substitution and then the functions are examined.
2. Mutants obtained via single amino acid substitution of each residue portion are ranked in order of desirability of functions. The mutations of the top three mutants excluding substitutions with cysteine or lysine are carried out in combination. The mutations of the top three mutants are selected again and carried out in combination with the mutations of the top three mutants obtained via single amino acid substitutions of the other sites (excluding substitutions with cysteine or lysine).
3. This procedure is repeated until all cysteine residue portions and lysine residue portions are substituted with other amino acids.

More specifically, the procedure is carried out as follows.

It is assumed that there are “n (number)” lysine and cysteine residues in a natural protein with a full-length of “m (number)” amino acids. The position of each residue on the amino acid sequence is determined to be Ai (i=1 to n).

The thus obtained mutation is represented by A1/MA1.

Regarding lysine and cysteine residues represented by Ai (i=2 to n) at other sites, a mutant gene is prepared by substituting codons encoding lysine and cysteine residues with codons encoding the above “amino acids other than lysine or cysteine” (maximum 18 types). The mutant gene is expressed and then the enzyme activity of the thus obtained double mutant enzyme protein is examined.

When the activity of the double mutants is examined, mutants exhibiting activity equivalent to or higher than that of the natural protein are observed. Up to three double mutants are selected from the double mutants in decreasing order of activity.

Next, triple mutants (maximum 3×18=54 types) are prepared by substituting lysine and cysteine residues of A3 of each of the thus obtained double mutants with amino acids (maximum 18 types) other than lysine and cysteine residues. The enzyme activity is then examined.

When the activity of triple mutants is examined, mutants exhibiting activity equivalent to or higher than that of the natural protein are observed.

Hereinafter, fourfold, n-fold mutants are prepared similarly. The final n-fold mutant is a target protein containing neither a lysine residue nor a cysteine residue.

With this procedure, a protein at least having functions equivalent to those of the original natural protein can be obtained. The phrase “functions equivalent to those of the original natural protein” means that the activity of the protein obtained via sequence alteration remains unchanged in terms of quality and is not lowered significantly in terms of amount compared with the original natural protein. For example, when an original natural protein is an enzyme that catalyzes a specific reaction, the protein obtained via sequence alteration also has enzyme activity that catalyzes the same reaction. Alternatively, when an original natural protein is an antibody that binds to a specific antigen, the protein obtained via sequence alteration has activity of an antibody capable of binding to the same antigen. The activity of a protein obtained via amino acid sequence alteration accounts for 10% or more, preferably 50% or more, more preferably 75% or more, further more preferably 90% or more, and particularly preferably 100% or more of the activity of the original natural protein. In the case of an enzyme, activity is represented by specific activity, for example. In the case of a protein capable of binding to another substance such as an antibody, activity is represented by binding ability. Methods for measuring such activity can be adequately selected depending on proteins.

As demonstrated in Examples described later, when partial sequences of different natural proteins capable of binding to antibody molecules are converted to sequences containing neither a cysteine residue nor a lysine residue, the converted partial sequences have functions equivalent to those of the partial sequence derived from natural proteins. This indicates the presence of a protein that comprises an amino acid sequence altered to be composed of 18 types of amino acid containing neither a cysteine residue nor a lysine residue based on the amino acid sequence of a natural protein having specific functions and retains functions equivalent to those of the naturally existing protein. This also suggests the universality of the present invention such that the present invention is applicable to all proteins. Also, it is predicted that a protein having target functions can be prepared by a de novo design technique or the like that involves artificially designing such a protein from an amino acid sequence and then synthesizing the protein. It is also suggested herein that a functional protein can be prepared via limitation such that 18 types of amino acid alone (containing neither a cysteine residue nor a lysine residue) are used in the de novo design technique, for example. It is also suggested herein that not only alteration of the amino acid sequence of a naturally derived protein, but also design and preparation of a novel functional protein having specific functions, which can be used as the R1 portion of the present invention, are possible.

Examples of the protein of the R1 portion include a protein having enzyme activity and a protein capable of binding to an antibody molecule. Known examples of a protein capable of binding to an antibody molecule include protein A derived from Staphylococcus aureus (disclosed in A. Forsgren and J. Sjoquist, J. Immunol. (1966) 97, 822-827), protein G derived from Streptococus sp. Group C/G (disclosed in the specification of EP Application (published) No. 1173239774906_{—}0 (1983)), protein L derived from Peptostreptococcus magnus (disclosed in the specification of U.S. Pat. No. 5,965,390 (1992)), protein H derived from group A Streptococcus (disclosed in the specification of U.S. Pat. No. 5,180,810 (1993)), protein D derived from Haemophilus influenzae (disclosed in the specification of U.S. Pat. No. 6,025,484 (1990)), protein Arp (Protein Arp4) derived from Streptococcus AP4 (disclosed in the specification of U.S. Pat. No. 5,210,183 (1987)), Streptococcal FcRc derived from group C Streptococcus (disclosed in the specification of U.S. Pat. No. 4,900,660 (1985)), a protein derived from group A streptococcus, Type II strain (U.S. Pat. No. 5,556,944 (1991)), a protein derived from Human Colonic Mucosal Epithelial Cell (disclosed in the specification of U.S. Pat. No. 6,271,362 (1994)), a protein derived from Staphylococcus aureus, strain 8325-4 (disclosed in the specification of U.S. Pat. No. 6,548,639 (1997)), and a protein derived from Pseudomonas maltophilia (disclosed in the specification of U.S. Pat. No. 5,245,016 (1991)).

The sequence shown in later-described Example 1 is the sequence (SEQ ID NO: 7) derived from the A domain of Staphylococcus-derived protein A, as shown below,

Ala-Asp-Asn-Asn-Phe-Asn-Lys-Glu-Gln-Gln-Asn-Ala-
Phe-Tyr-Glu-Ile-Leu-Asn-Met-Pro-Asn-Leu-Asn-Glu-
Glu-Gln-Arg-Asn-Gly-Phe-Ile-Gln-Ser-Leu-Lys-Asp-
Asp-Pro-Ser-Gln-Ser-Ala-Asn-Leu-Leu-Ser-Glu-Ala-
Lys-Lys-Leu-Asn-Glu-Ser-Gln-Ala-Pro-Lys

(originally containing no cysteine residue). It was demonstrated that through alteration of this sequence, a protein containing neither a cysteine residue nor a lysine residue and having immunoglobulin (IgG)-binding activity equivalent to that of the naturally derived protein comprising the above amino acid sequence can be obtained.

The sequence shown in later-described Example 2 is the sequence (SEQ ID NO: 8) derived from the G1 domain of Streptococcus-derived protein G, as shown below,

Thr-Tyr-Lys-Leu-Ile-Leu-Asn-Gly-Lys-Thr-Leu-Lys-
Gly-Glu-Thr-Thr-Thr-Glu-Ala-Val-Asp-Ala-Ala-Thr-
Ala-Glu-Lys-Val-Phe-Lys-Gln-Tyr-Ala-Asn-Asp-Asn-
Gly-Val-Asp-Gly-Glu-Trp-Thr-Tyr-Asp-Asp-Ala-Thr-
Lys-Thr-Phe-Thr-Val-Thr-Glu-Arg-Pro-Glu-Val-Ile-
Asp-Ala-Ser-Glu-Leu-Thr-Pro-Ala-Val-Thr

(originally containing no cysteine residue). It was demonstrated that through alteration of this sequence, a protein containing neither a cysteine residue nor a lysine residue and having IgG binding activity equivalent to that of the naturally derived protein comprising the above amino acid sequence can be obtained.

The sequence shown in later-described Example 3, is the sequence (SEQ ID NO: 9) derived from the B1 domain of Peptostreptococcus-derived protein L, as shown below,

Val-Thr-Ile-Lys-Ala-Asn-Leu-Ile-Tyr-Ala-Asp-Gly-
Lys-Thr-Gln-Thr-Ala-Glu-Phe-Lys-Gly-Thr-Phe-Glu-
Glu-Ala-Thr-Ala-Glu-Ala-Tyr-Arg-Tyr-Ala-Asp-Leu-
Leu-Ala-Lys-Glu-Asn-Gly-Lys-Tyr-Thr-Val-Asp-Val-
Ala-Asp-Lys-Gly-Tyr-Thr-Leu-Asn-Ile-Lys-Phe-Ala

(originally containing no cysteine residue). It was demonstrated that through alteration of this sequence, a protein containing neither a cysteine residue nor a lysine residue and having IgG binding activity equivalent to that of the naturally derived protein comprising the above amino acid sequence can be obtained.

In addition, random mutagenesis is generally employed in many cases as a method for causing mutation in the amino acid sequence of a natural protein. Moreover, a phage display method is employed in many cases for selection of functions. However, as long as such methods are employed, a possibility of obtaining an altered protein that comprises an amino acid sequence containing neither a cysteine residue nor a lysine residue and having functions equivalent to those of the natural protein is significantly low. Hence, a sequence corresponding to the R1 portion of the present invention cannot be obtained. The above method developed by the present inventors makes it possible to obtain a sequence corresponding to the R1 portion of the present invention.

Furthermore, when the sequence of the R1 portion is a sequence comprising two portions represented by P-Q, the sequence of the P portion is represented by (Ser or Ala)-(Gly) n (where n denotes an arbitrary integer ranging from 1 to 10). An example of the sequence is Ser-Gly-Gly-Gly-Gly (SEQ ID NO: 23). Also, the sequence of the Q portion is the sequence of a protein having a repeating unit, wherein a sequence unit containing neither a lysine residue nor a cysteine residue is repeated. Examples of such sequence are as listed below.

A protein described later in Example 5 contains the sequence of the Q portion in which a sequence unit prepared by altering a sequence derived from the A domain of Staphylococcus-derived protein A so as to contain neither a lysine residue nor a cysteine residue is repeated.

(Ala-Asp-Asn-Asn-Phe-Asn-Arg-Glu-Gln-Gln-Asn-Ala-Phe-Tyr-Glu-Ile-Leu-Asn-Met-Pro-Asn-Leu-Asn-Glu-Glu-Gln-Arg-Asn-Gly-Phe-Ile-Gln-Ser-Leu-Arg-Asp-Asp-Pro-Ser-Gln-Ser-Ala-Asn-Leu-Leu-Ser-Glu-Ala-Arg-Arg-Leu-Asn-Glu-Ser-Gln-Ala-Pro-Gly) n (where n denotes an arbitrary integer ranging from 2 to 5 and the sequence shown in parentheses is represented by SEQ ID NO: 24)

Such protein in which a sequence unit is repeated exerted IgG binding activity far greater than that exerted by a protein containing no such repetition.

A protein described later in Example 6 contains the sequence of the Q portion in which a sequence unit prepared by altering a sequence derived from the G1 domain of Streptococcus-derived protein G so as to contain neither a lysine residue nor a cysteine residue is repeated.

(Ala-Tyr-Arg-Leu-Ile-Leu-Asn-Gly-Arg-Thr-Leu-Arg-Gly-Glu-Thr-Thr-Thr-Glu-Ala-Val-Asp-Ala-Ala-Thr-Ala-Glu-Arg-Val-Phe-Arg-Gln-Tyr-Ala-Asn-Asp-Asn-Gly-Val-Asp-Gly-Glu-Trp-Thr-Tyr-Asp-Asp-Ala-Thr-Arg-Thr-Phe-Thr-Val-Thr-Glu-Arg-Pro-Glu-Val-Ile-Asp-Ala-Ser-Glu-Leu-Thr-Pro-Ala-Val-Thr-Pro-Gly) n (where n denotes an arbitrary integer ranging from 2 to 5 and the sequence shown in parentheses is represented by SEQ ID NO: 25)

Such protein having a repeating sequence unit exerted IgG binding activity far greater than that exerted by a protein containing no such repetition.

A protein described later in Example 7 contains the sequence of the Q portion in which a sequence unit prepared by altering a sequence derived from the B1 domain of Peptostreptococcus-derived protein L so as to contain neither a lysine residue nor a cysteine residue is repeated.

(Ala-Thr-Ile-Arg-Ala-Asn-Leu-Ile-Tyr-Ala Asp-Gly-Arg-Thr-Gln-Thr-Ala-Glu-Phe-Arg Gly-Thr-Phe-Glu-Glu-Ala-Thr-Ala-Glu-Ala Tyr-Arg-Tyr-Ala-Asp-Leu-Leu-Ala-Arg-Glu Asn-Gly-Arg-Tyr-Thr-Val-Asp-Val-Ala-Asp Arg-Gly-Tyr-Thr-Leu-Asn-Ile-Arg-Phe-Ala Pro-Gly-) n (where n denotes an arbitrary integer ranging from 2 to 5 and the sequence shown in parentheses is represented by SEQ ID NO: 26)

Such protein in which a sequence unit is repeated exerted IgG binding activity far greater than that exerted by a protein containing no such repetition.

The R2 portion is an arbitrary spacer sequence composed of amino acid residues other than lysine and cysteine residues. The spacer sequence is immobilized together with the R1 portion on an immobilization carrier. The R2 portion is characterized by containing neither a lysine residue nor a cysteine residue. In general, when a protein is immobilized, a protein that has its unique functions and is a subject for immobilization is immobilized. When a protein alone is immobilized, the functions of the immobilized protein may be inhibited by steric hindrance or the like with an immobilization carrier. In the case of the present invention, the R2 portion plays a role as an appropriate linker to prevent the functions of the R1 portion from being inhibited by binding to an immobilization carrier upon immobilization. The role as a linker is to keep an appropriate distance between a protein having the specific functions of the R1 portion and an immobilization carrier. Therefore, the R2 portion is required to be an arbitrary amino acid sequence with a fixed length and inert. In the present invention, only the R3 portion requires the sole cysteine residue for immobilization reaction. Also, a primary amine is used as a functional group for binding an immobilization carrier with an immobilization protein. Accordingly, a lysine residue having a primary amine group in its side chain is inappropriate as an amino acid residue composing a linker. Hence, the R2 portion should be composed of 18 types of amino acid residue other than cysteine and lysine residues.

In addition, when the functions of the protein of the R1 portion are not inhibited even if the R1 portion is directly bound to an immobilization carrier, the R2 portion may be absent. In this case, the above general formula is also represented by R1-R3-R4-R5. The number of amino acids of the R2 portion is not limited and may be 0 (that is, absent) or range from 1 to 10 amino acids, and preferably range from 2 to 5 amino acids. An example of the sequence of the R2 portion is polyglycine or the like comprising 1 to 10 or 2 to 5 glycines. In later-described Examples, an example using a chain of the most simple amino acid, glycine, R2=Gly-Gly-Gly-Gly (SEQ ID NO: 16) is presented, but the example in the present invention is not limited thereto.

The above protein comprising the amino acid sequence represented by the general formula R1-R2-R3-R4-R5 is characterized by having the sole cysteine residue in the R3 sequence portion alone. Therefore, an SH group that is a functional group in the side chain of the sole cysteine residue is cyanated to give a cyanocysteine residue. Through a reaction between the cyanocysteine residue and a primary amine on an immobilization carrier, only the portion represented by R1-R2 of the above amino acid sequence represented by the general formula R1-R2-R3-R4-R5 can be immobilized on the immobilization carrier in an orientation-controlled manner. In a cyanocysteine-mediated immobilization reaction, the sequence of the R4 portion; that is, a sequence richly containing acidic amino acids that acidify the isoelectric point of a protein comprising the entire sequence is contained, so that the entire protein comprising the amino acid sequence represented by the general formula R1-R2-R3-R4-R5 can be negatively charged. As a result, the above protein can be immediately bound adsorptively to an immobilization carrier having a positively-charged primary amine via electrostatic interactions. Then, the subsequent mild reaction that is a cyanocysteine-mediated binding reaction can be efficiently caused to proceed. Binding of such protein to an immobilization carrier proceeds as described above, so that high-density immobilization becomes possible. In addition, when the isoelectric point of a protein comprising the amino acid sequence represented by R1-R2-R3-R5 or R1-R3-R5 excluding the R4 portion is acidic, R4 may be absent.

An example of the sequence of the R3 portion is an amino acid sequence comprising two amino acids represented by cysteine-X (where X denotes an amino acid other than lysine or cysteine). X is not limited. However, when a protein comprising the amino acid sequence represented by R1-R2 is immobilized using the polypeptide of the present invention comprising the amino acid sequence represented by the general formula R1-R2-R3-R4-R5, cysteine of the R3 portion is converted to cyanocysteine. At this time, the amino acid next to the cyanocysteine is converted to alanine, so that a cyanocysteine-residue-mediated amide bond forming reaction is likely to take place. Hence, X is preferably alanine. Discovery of a cyanocysteine-mediated binding reaction and the analysis of the reaction are described in T. Takenawa, et al. (1998) J. Biochem. 123, 1137-1144 or Y. Ishihama et al. (1999) Tetrahedron Lett. 40, 3415-3418, for example.

A sequence preferred as that of the R4 portion contains acidic amino acid residues capable of acidifying the isoelectric point of the entire protein comprising the amino acid sequence represented by the general formula R1-R2-R3-R4-R5. Here, the phrase “sequence containing an acidic amino acid residue(s) capable of acidifying the isoelectric point of the entire protein” refers to a sequence containing such acidic amino acid residues sufficiently to acidify the isoelectric point of the entire protein in terms of the type and the number thereof. The sequence of the R4 portion preferably contains a high aspartic acid or glutamic acid content. The isoelectric point of a protein depends on the types and the numbers of constituent amino acids. For example, when many basic amino acids such as lysine and arginine are contained, the number of aspartic acids and glutamic acids is required to be greater than the total number of basic amino acids. Persons skilled in the art can easily predict the isoelectric point of a protein by calculation. Preferably, a sequence containing a high aspartic acid or glutamic acid content is designed so that the isoelectric point of the above protein comprising the amino acid sequence of the general formula R1-R2-R3-R4-R5 is a value between 4 and 5. The number of amino acids in the sequence of the R4 portion is not limited and may be 0 (that is, absent) or range from 1 to 20, preferably 1 to 10, or 1 to 20, and may preferably range from 1 to 10. An example is a polyaspartic acid comprising 1 to 10 aspartic acids.

The R5 portion is a sequence portion that is used for purifying a synthesized protein comprising the amino acid sequence represented by the general formula R1-R2-R3-R4-R5. An example of the sequence of the R5 portion is a sequence capable of binding to a specific compound; that is, an affinity tag sequence. When a protein containing such tag is purified using an antibody specific to the tag, an epitope tag may also be an example. An example of such an affinity tag sequence is a polyhistidine sequence comprising 2 to 12, preferably 4 or more, more preferably 4 to 7, further more preferably 5 or 6 histidines. In this case, the above polypeptide can be purified by nickel chelate column chromatography using nickel as a ligand. Also, the polypeptide can be purified by affinity chromatography using a column to which an antibody against polyhistidine has been immobilized as a ligand. In addition to such tags, a HAT tag, a HN tag, and the like comprising histidine-containing sequences can also be used. Examples of tags to be used for the R5 portion and ligands to be used for affinity chromatography are as listed below, but the examples are not limited thereto. All known affinity tags (epitope tags) can be used herein. Other examples of affinity tags include a V5 tag, an Xpress tag, an AU1 tag, a T7 tag, a VSV-G tag, a DDDDK tag, an S tag, CruzTag09, CruzTag 22, CruzTag41, a Glu-Glu tag, a Ha.11 tag, and a KT3 tag.

| | |
|---|---|
| Tag of the R5 portion | ligand |
| Glutathione-S-transferase (GST) | glutathione |
| Maltose binding protein (MBP) | amylose |
| HQ tag | nickel |
| (HQHQHQ; SEQ ID NO: 17) | |
| Myc tag | anti-Myc antibody |
| (EQKLISEEDL; SEQ ID NO: 18) | |
| HA tag | anti-HA antibody |
| (YPYDVPDYA; SEQ ID NO: 19) | |
| FLAG tag | anti-FLAG antibody |
| (DYKDDDDK; SEQ ID NO: 20) | |

As a result of immobilization reaction using a protein comprising the amino acid sequence represented by the general formula R1-R2-R3-R4-R5, the sequence portion of R3-R4-R5 is excised and remains in the reaction solution. Hence, the portion of R3-R4-R5 can be removed by appropriate washing after the immobilization reaction. At this time, the portion can also be removed using the affinity tag of the R5 portion. Accordingly, the properties of the sequence portion of R3-R4-R5 have no effect on the functions and the like of an immobilized protein. The portion of R3-R4-R5 can be any sequence as long as it satisfies the above conditions.

Examples of the combinations of R3, R4, and R5 are as follows,

### R3=Cys-Ala,

### R4=Asp-Asp-Asp-Asp-Asp-Asp (SEQ ID NO: 21), and

### R5=His-His-His-His-His-His (SEQ ID NO: 22).

The examples of the present invention are not limited thereto.

The present invention also encompasses a method for designing and preparing a protein comprising the amino acid sequence represented by the general formula R1-R2-R3-R4-R5, in order to immobilize an arbitrary subject protein (to be immobilized) on an immobilization carrier in accordance with conditions that each of the R1, R2, R3, R4, and R5 portions should satisfy.

For example, the method for designing or preparing such protein comprises the following steps (a) to (e) of:

(a) selecting as the sequence of the R1 portion the sequence of a subject protein to be immobilized, which contains neither a lysine residue nor a cysteine residue;
(b) not selecting the sequence of the R2 portion when the sequence of the R2 portion is absent or selecting a spacer sequence composed of amino acid residues other than lysine and cysteine residues when the sequence of the R2 portion is present;
(c) selecting as the sequence of the R3 portion a sequence composed of two residues of amino acid represented by cysteine-X (where X denotes an amino acid residue other than lysine or cysteine);
(d) not selecting the sequence of the R4 portion when the sequence of the R4 portion is absent or selecting a sequence containing neither a lysine residue nor a cysteine residue, but containing an acidic amino acid residue capable of acidifying the isoelectric point of the entire protein comprising the amino acid sequence represented by the general formula R1-R2-R3-R4-R5 when the sequence of the R4 portion is present; and
(e) selecting as the sequence of the R5 portion an affinity tag sequence for protein purification.

Moreover, when the R1 portion is represented by P-Q, the sequence of the P portion may be absent. When the sequence of the P portion is present, a sequence comprising (Ser or Ala)-(Gly) n (where n denotes an arbitrary integer ranging from 1 to 10) is selected. As the sequence of the Q portion, the sequence of a protein having a repeating unit is selected, in which a sequence unit containing neither a lysine residue nor a cysteine residue is repeated.

A protein comprising the amino acid sequence represented by the general formula R1-R2-R3-R4-R5 can be chemically synthesized based on the amino acid sequence. Moreover, a DNA sequence encoding the protein comprising the amino acid sequence represented by the general formula R1-R2-R3-R4-R5 can be prepared by chemical synthesis or the like. A portion thereof can also be prepared from a naturally derived gene via amplification by the PCR method, separation, recombination, and the like. A sequence required for transcriptional initiation and a sequence required for translational initiation are ligated upstream of the thus prepared DNA sequence encoding the amino acid sequence represented by the general formula R1-R2-R3-R4-R5 and a stop codon is further ligated downstream of the same, so that a DNA sequence is prepared. This DNA sequence is incorporated into an appropriate vector DNA for transduction into hosts. The DNA sequence is expressed within the hosts, so that a target protein comprising the amino acid sequence represented by the general formula R1-R2-R3-R4-R5 can be prepared.

The thus prepared protein comprising the amino acid sequence represented by the general formula R1-R2-R3-R4-R5 can be separated and purified from the cell-free extracts of hosts expressing the protein with the use of the sequence of the R5 portion as described above. At this time, the same sequence (e.g., a polyhistidine sequence) is always used as the sequence of the R5 portion regardless of the sequence of the R1 portion, a common purification and separation method can be applied to an arbitrary protein comprising the amino acid sequence represented by the general formula R1-R2-R3-R4-R5.

The present invention further encompasses a protein comprising the amino acid sequence represented by the general formula R2-R3-R4-R5, to which the amino acid sequence R1 of a subject protein (to be immobilized) is ligated (specifically, the sequence R1 is ligated to the N-terminal side of the R2 portion), so that a protein comprising the amino acid sequence represented by the general formula R1-R2-R3-R4-R5 can be prepared. Furthermore, the present invention also encompasses a DNA encoding the amino acid sequence represented by the general formula R2-R3-R4-R5, to which the nucleotide sequence of a DNA encoding a subject protein to be immobilized (amino acid sequence R1) is ligated (specifically, the DNA encoding the subject protein is ligated to the 5′ terminal side of the DNA encoding the amino acid sequence represented by the general formula R2-R3-R4-R5), so that a DNA encoding the amino acid sequence represented by the general formula R1-R2-R3-R4-R5 can be prepared. Such protein comprising the amino acid sequence represented by the general formula R2-R3-R4-R5 or a DNA encoding the amino acid sequence represented by the general formula R2-R3-R4-R5 can be used as an amino acid sequence or a nucleotide sequence for a commonly employed technique for preparation of a protein for immobilization and particularly for preparation of a protein comprising the amino acid sequence represented by the general formula R1-R2-R3-R4-R5 to which an arbitrary subject protein to be immobilized has been ligated. In this case, since the R5 portion is common to all cases, a protein for immobilization can be purified by the same technique regardless of the sequence of the R1 portion.

A subject protein to be immobilized can be immobilized on a carrier using the protein for immobilization of the present invention comprising the amino acid sequence represented by the general formula R1-R2-R3-R4-R5 according to methods disclosed in JP Patent No. 3788828, JP Patent No. 2990271, JP Patent No. 3047020, JP Patent Publication (Kokai) No. 2003-344396 A, and the like. Specifically, the R1-R2 portion is immobilized on an immobilization carrier by converting the cysteine residue of the R3 portion of the protein of the present invention comprising the amino acid sequence represented by the general formula R1-R2-R3-R4-R5 to cyanocysteine through cyanation and then reacting the protein having cyanocysteine with the immobilization carrier having a primary amino group represented by the general formula “NH2-Y” (where Y denotes an arbitrary immobilization carrier) as a functional group under weak alkaline conditions (pH 8 to 10). The resultant prepared by binding the R1-R2 portion to the immobilization carrier is represented by R1-R2-CO—NH—Y (where Y denotes the same as defined above), wherein the R1-R2 portion is bound to the immobilization carrier only through the carboxy terminus of the R2 portion. In addition, when the above protein for immobilization contains no R2 portion, the protein is represented by R1-CO—NH—Y (where Y denotes the same as defined above). A cyanation reaction can be carried out using a cyanation reagent. Examples of such a cyanation reagent that can be used herein include 2-nitro-5-thiocyanobenzoic acid (NTCB) (see Y. Degani, A. Ptchornik, Biochemistry, 13, 1-11 (1974)) and 1-cyano-4-dimethylaminopyridinium tetrafluoroborate (CDAP).

Furthermore, the method disclosed in JP Patent Publication (Kokai) No. 2003-344396 A comprises causing an immobilization carrier to adsorb a protein comprising the amino acid sequence represented by the general formula R1-R2-R3-R4-R5, carrying out cyanation of the cysteine residue so that the above reaction is performed, and then immobilizing a protein comprising the amino acid sequence represented by R1-R2 on the immobilization carrier. To cause an immobilization carrier to adsorb a protein, such reaction between the protein and the immobilization carrier is carried out under neutral to weak alkaline conditions (pH 7 to 10). Under weak alkaline reaction conditions, a protein is negatively charged while an immobilization carrier is positively charged and then they are mutually adsorbed and bound via electrostatic interactions. The present invention also encompasses an immobilization carrier caused to adsorb a protein comprising the amino acid sequence represented by the general formula R1-R2-R3-R4-R5. Many unreacted primary amines are present in an immobilization carrier portion in the case of a protein immobilization carrier (to which the R1-R2 portion is immobilized) prepared by cyanocysteine-mediated immobilization reaction using the protein of the present invention comprising the amino acid sequence represented by the general formula R1-R2-R3-R4-R5. When a lysine residue or a cysteine residue is present in the thus immobilized protein, the remaining active amines can limit the use of immobilized proteins of the present invention. However, the protein portion immobilized by the method of the present invention contains neither a lysine residue nor a cysteine residue. Hence, the carrier surface on which the protein has been immobilized can be treated with a masking agent for a primary amine so that the protein portion is not affected by the remaining active amines. As a masking agent, acetic anhydride, maleic anhydride, and the like are preferred, but any masking agent can be used herein. Accordingly, the present invention is not limited by the types of masking agent.

The present invention further provides an immobilized protein and a carrier to which the protein has been immobilized (obtained by the above method) by firmly binding a protein comprising an amino acid sequence containing neither a cysteine residue nor a lysine residue to the immobilization carrier having a primary amino group via an appropriate linker sequence and the amide (peptide) bond.

Any immobilization carrier having a primary amino group can be used in the present invention, as long as it is an immobilization carrier having a primary amino group. Examples of “carrier” in the present invention include any carriers such as particulate carriers and plate-like or sheet-shaped substrates, as long as they are insoluble and proteins can be immobilized thereon. Examples of an “immobilization carrier” include “immobilization substrates.” Moreover, an “immobilization carrier” may also be referred to as an “insoluble carrier.” Examples of a commercially available carrier having a primary amino group include Amino-Cellulofine (marketed by Seikagaku Corporation), AF-Amino Toyopearl (marketed by TOSOH), EAH-Sepharose 4B and Lysine-Sepharose 4B (marketed by Amersham Biosciences), and Porus 20NH (marketed by Boehringer Mannheim). Also, a primary amino group is introduced onto glass beads, glass plates, or the like using a silane compound (e.g., 3-aminopropylmethoxysilane) that has a primary amino group and then the resultant can also be used.

Furthermore, the content of a primary amino group per unit volume of carrier can be increased by introducing a polymer compound that has a primary amino group in its repeating unit into an immobilization carrier (see JP Patent Publication (Kokai) No. 2004-345956 A).

For example, polyallylamine-grafted Cellulofine is known as a carrier prepared by introducing a polymer compound that has a primary amino group in its repeating unit into an immobilization carrier (paper for reference: see Ung-Jin Kim, Shigenori Kuga, Journal of Chromatography A, 946, 283-289 (2002)). Furthermore, CNBr-activated Sepharose FF, NHS-activated Sepharose FF, and a carrier having chemical reactivity to a primary amino group are known. A polymer compound such as polyallylamine having a primary amino group in its repeating unit is caused to act on such a carrier, so that the carrier to which the polymer compound is covalently bound can be prepared. At this time, the content of a primary amino group that can be used for immobilization reaction in a carrier to be prepared can be varied by adequately adjusting the mixing ratio of a polymer compound having a primary amino group in its repeating unit to an activation carrier.

Meanwhile, any polymer compound can be used herein, as long as it has a primary amino group and portions other than this are substantially inactive to a protein to be immobilized. As a commercially available polymer compound, polyallylamine, poly L-lysine, or the like can be used. Therefore, the present invention is not limited by the types of immobilization carrier.

### EXAMPLES

The present invention will be described in detail by examples as follows, but the present invention is not limited by these examples.

In the following Examples, experimental methods described below were used commonly.

### [Gene Synthesis]

Genes described in the Examples were synthesized by contracted manufacturers of synthetic genes, unless otherwise specified. dsDNA was synthesized based on a nucleotide sequence shown in each case and then inserted into the BamH I-EcoR I site of a pUC18 vector. The sequences of the thus obtained clones were confirmed by single strand analysis and then the nucleotide sequence information was verified. Sites for which mismatches had been confirmed were subjected to correction using a technique such as site directed mutagenesis, and then the thus obtained plasmid DNA (approximately 1 microgram) was introduced. Regarding the target portion in the plasmid introduced, the sequence was confirmed again by sequencing.

### [Preparation of Mutant by Single Amino Acid Substitution]

Amino acid substitution was carried out according to a QuickChange method (described for a QuickChange Site-Directed Mutagenesis kit, Stratagene) using a DNA primer prepared by converting a DNA sequence encoding an amino acid at a substitution site to a target codon sequence so that 24 bases of the original sequence were present on both of its ends and its complementary DNA primer.

### [Protein Purification]

Escherichia coli JM109 strain transformed with a recombinant plasmid was cultured overnight at 35° C. in 2 liters of medium (containing 20 g of sodium chloride, 20 g of yeast extract, 32 g of triptone, and 100 mg of ampicillin sodium). Subsequently, the culture solution was centrifuged at a low speed (5,000 rotations per minute) for 20 minutes, so that 3 g to 5 g of cells (wet weight) was obtained. This was suspended in 20 ml of 10 mM phosphate buffer (pH 7.0). The cells were disrupted with a French press and then centrifuged at a high speed for 20 minutes (20,000 rotations per minute), so that a supernatant was separated. Streptomycin sulfate was added to the thus obtained supernatant to a final concentration of 2%. After 20 minutes of stirring, the solution was centrifuged at a high speed (20,000 rotations per minute) for 20 minutes, so that a supernatant was separated. Subsequently, ammonium sulfate treatment was carried out. The thus obtained supernatant was applied to a nickel chelate column (purchased from GE Healthcare Bioscience). The column was sufficiently washed using 200 ml or more of washing buffer (5 mM imidazole, 20 mM sodium phosphate, 0.5 M sodium chloride; pH 7.4). After washing, 20 ml of elution buffer (0.5 M imidazole, 20 mM sodium phosphate, 0.5 M sodium chloride; pH 7.4) was applied, so that a target protein was eluted. Subsequently, to remove imidazole from the protein solution, dialysis was carried out against 5 liters of 10 mM phosphate buffer (pH 7.0). MWCO3500 (purchased from Spectrum Laboratories) was used as a dialysis membrane. After dialysis, the target protein was dried using a centrifugal vacuum dryer.

### [Analysis of Binding Properties to Human Antibody IgG Molecule]

A Biacore surface plasmon resonance biosensor (Biacore) was used for analyzing the binding properties of target proteins, and the analysis was carried out according to protocols provided by Biacore.

Running buffer with a composition of 10 mM HEPES (pH 7.4), 150 mM sodium chloride, 5 μM EDTA, and 0.005% Surfactant P20 (Biacore), which had been deaerated in advance, was used.

As a sensor chip, a Sensor Chip NTA (Biacore) was used. A sensor chip was sufficiently equilibrated with the running buffer and then a 5 mM nickel chloride solution was injected thereinto, so that arrangement of nickel ions was completed. Subsequently, the recombinant protein was immobilized on the sensor chip by injection of the recombinant protein solution (in the running buffer with a concentration of 100 μg/mL).

The binding reaction between the immobilized recombinant protein and human IgG was carried out as follows. Human IgG (Sigma-Aldrich Corporation) solutions were diluted and prepared to give 7 types of concentration ranging from 0.25 μg/mL to 20 μg/mL using running buffer. Each solution was injected sequentially followed by injection of the running buffer, so as to keep the solution flowing. The association and dissociation phenomena of the antibody were quantitatively observed. In addition, the flow of the solution flowing was 20 μL/min, the time for observing binding (the time for injecting an antibody solution) was 4 minutes, and the time for observing dissociation was 4 minutes. After injection of the antibody solution with each concentration and the following observation of the phenomena of association and dissociation, a 6 M guanidine hydrochloride solution was subsequently injected for 3 minutes. Thus, all human IgGs binding to the immobilized recombinant proteins were released and then regenerated using running buffer, so that they could be used for the subsequent measurements.

Changes in mass over time on the surface plasmon resonance sensor surfaces observed were measured using RU (the unit defined by Biacore) and then association rate constants (kass), dissociation rate constants (kdis), and dissociation constants (Kd=kass/kdis) were found.

### [Immobilization of Recombinant Protein]

Each protein was dialyzed in advance for 3 or more times against 10 mM phosphate buffer (pH 8.0) containing a 1000-fold-volume 5 mM ethylenediamine tetraacetate (EDTA). Each dialyzed protein sample was diluted with the same buffer as that used for dialysis. Thus, protein samples with various concentrations were prepared.

The thus prepared proteins for immobilization were each mixed with Amino-Cellulofine (amine content: 20 μmoles, NH₂/ml) and then the mixture was mildly stirred for 2 hours or more at room temperature.

The SH group of cysteine of the adsorbed protein was cyanated by suspending the carrier comprising the protein adsorptively immobilized thereon in a 10 mM phosphate buffer (pH 7.0) containing 5 mM EDTA, adding 2-nitro-5-thiocyanobenzoic acid (NTCB) to a final concentration of 5 mM, and allowing the reaction to proceed at room temperature for 4 hours. Thereafter, centrifugation was carried out at 1000 rpm for several seconds, the carrier was submerged to remove the supernatant, followed by suspension in a 10 mM phosphate buffer (pH 7.0). This procedure was repeated 5 times to eliminate NTCB, and the like.

The cyanated adsorptively immobilized protein was centrifuged at 1000 rpm for several seconds, the carrier was submerged, and then the supernatant was removed. The resultant was suspended in a 10 mM borate buffer (pH 9.5) containing 5 mM EDTA, followed by mild stirring at room temperature for 24 hours or more. Thus, the immobilization reaction was carried out. Thereafter, centrifugation was carried out at 1000 rpm for several seconds. The carrier was submerged and the supernatant was removed, followed by suspension in a 10 mM phosphate buffer (pH 8.0) containing 1M KCl. This procedure was repeated 5 times to eliminate the by-product of the immobilization reaction.

Unreacted primary amine on the immobilization carrier was acetylated using acetic anhydride. Such immobilized protein contains no lysine residue, so that modifications other than aminoterminal acetylation do not take place because of acetylation. In general, amino termini hardly contribute to the binding activity. Thus, it is considered that the hypofunction of the immobilized ligand protein due to this procedure can be ignored.

### [Measurement of IgG Binding Capacity of Immobilization Carrier]

An immobilization carrier (10 μl) and 990 μl of human IgG (2 mg) were mixed in 10 mM phosphate buffer (pH 7.0), followed by 12 hours of mild stirring at room temperature. The resultant was washed 5 times with 2 ml of 10 mM phosphate buffer (pH 7.0) containing 1 M KCl. Absorbance at 280 nm was measured, so as to confirm that no protein was contained in the final wash fluid.

Immunoglobulin G was released from the carrier by adding 1 ml of 0.1 M acetic acid solution to the immobilization carrier collected by centrifugation after washing. The amount of human IgG released in the solution was measured by measuring absorbance at 280 nm and then using the absorption coefficient (E2801%=14.0). The result was divided by the amount of the carrier used, so as to find the IgG binding capacity (mg/ml carrier).

### Example 1

### Conversion to a Sequence Containing Neither a Cysteine Residue Nor a Lysine Residue and to a Sequence for Immobilization Based on a Sequence Derived from Domain A of Staphylococcus-Derived Protein A

The sequence derived from domain A of Staphylococcus-derived protein A is represented by SEQ ID NO: 7.

Based on the amino acid sequence represented by SEQ ID NO: 7, the following DNA sequence (SEQ ID NO: 11):

GGATCCTTGA CAATATCTTA ACTATCTGTT ATAATATATT
GACCAGGTTA ACTAACTAAG CAGCAAAAGG AGGAACGACT
ATGGCTGATA ACAATTTCAA CAAAGAACAA CAAAATGCTT
TCTATGAAAT CTTGAATATG CCTAACTTAA ACGAAGAACA
ACGCAATGGT TTCATCCAAA GCTTAAAAGA TGACCCAAGC
CAAAGTGCTA ACCTATTGTC AGAAGCTAAA AAGTTAAATG
AATCTCAAGC ACCGAAAGGT GGCGGTGGCT GCGCTGATGA
CGATGACGAT GACCATCATC ACCACCATCA TTAAGAATTC
C

was designed and synthesized, so that a sequence encoding the amino acid sequence (SEQ ID NO: 10) represented by:

Met-Ala-Asp-Asn-Asn-Phe-Asn-Lys-Glu-Gln-Gln-Asn-
Ala-Phe-Tyr-Glu-Ile-Leu-Asn-Met-Pro-Asn-Leu-Asn-
Glu-Glu-Gln-Arg-Asn-Gly-Phe-Ile-Gln-Ser-Leu-Lys-
Asp-Asp-Pro-Ser-Gln-Ser-Ala-Asn-Leu-Leu-Ser-Glu-
Ala-Lys-Lys-Leu-Asn-Glu-Ser-Gln-Ala-Pro-Gly-Gly-
Gly-Gly-Gly-Cys-Ala-Asp-Asp-Asp-Asp-Asp-His-His-
His-His-His-His

had appropriate transcription initiation functions, appropriate translation initiation functions, and a restriction enzyme sequence for incorporation into a vector.

pPAA was prepared by inserting the sequence represented by SEQ ID NO: 11 into the BamH I-EcoR I site of a pUC18 vector.

The protein was separated and purified from the Escherichia coli JM109 strain transformed with pPAA according to the above-described method. As a result, the target protein was obtained with a yield of approximately 150 mg/2 L of culture solution. The obtained protein was subjected to amino terminal sequence analysis and mass number analysis, so that the amino terminus was found to be alanine and the mass number of the obtained purified protein was found to be 8,540 daltons, as measured using a mass spectrometer. It was confirmed that the obtained protein had been subjected to amino-terminal processing of methione residue corresponding to the initiation codon, as generally observed when a recombinant protein with a sequence containing methionine-alanine as the amino terminal sequence is expressed by Escherichia coli.

Next, the 7^{th}, 35^{th}, 49^{th}, 50^{th}, and 58^{th} lysine residues from the amino terminus in SEQ ID NO: 7 were subjected to amino acid substitution. Specifically, first, the 58^{th} lysine was substituted with glycine to prepare a mutant. Amino acid substitution was carried out as follows. AAA, the DNA sequence encoding the 58^{th} lysine, was converted to GTT. Mutation was carried out according to the QuickChange method (described for a QuickChange Site-Directed Mutagenesis kit (Stratagene)) using a DNA primer having 24 bases of the original sequence on both ends, its complementary DNA primer, and pPAA as a template. Thus, a plasmid having the target mutation was separated (designated as pPAA-K58G).

Furthermore, amino acid substitution of the 7^{th} lysine was carried out as follows using pPAA-K58G as a template. DNAs encoding lysine residues were each converted to CGT codons so that a DNA primer and its complementary DNA were synthesized. With the use thereof as primers, a mutant was prepared by the QuickChange method. Thus, a plasmid having the target mutation was prepared (designated as pPAA-RKKKG). With the use of such plasmid as a template, a plasmid expressing a mutant in which the 35^{th} lysine residue had been converted to an arginine residue (designated as pPAA-RRKKG), a plasmid expressing a mutant in which the 49^{th} lysine residue had been converted to an arginine residue (designated as pPAA-RRRKG), a plasmid expressing a mutant in which the 50^{th} lysine residue had been converted to an arginine residue (designated as pPAA-RRRRG), and so on were prepared. The finally obtained recombinant plasmid pPAA-RRRRG was a plasmid expressing a protein A fragment mutant, comprising a sequence in which all lysine residues in the wild-type-derived protein fragment sequence had been converted to arginine or glycine (that is, the sequence represented by SEQ ID NO: 1).

Escherichia coli transformed with the recombinant plasmid pPAA-RRRRG expressed a protein comprising the sequence represented by SEQ ID NO: 1. The recombinant protein was homogenously purified in a manner similar to the above method, specifically by culturing of Escherichia coli, cell disruption, pretreatment, and procedures for nickel chelate column chromatography.

Moreover, with the use of the recombinant plasmid pPAA-RRRRG as a template, the 7^{th}, 35^{th}, 49^{th}, and 58^{th} amino acid residues were each subjected to single amino acid substitution, thereby preparing various mutants. The proteins were prepared.

The various thus obtained proteins were examined using Biacore in terms of their activity to bind to human IgG

Table 1 shows the results.

Other than the wild type sequence, all sequences were derived from A domain of protein A containing neither cysteine nor lysine. It was revealed that if the types of amino acid residue to be used herein are limited, the resultants retain the activity to bind to human IgG.

It was also revealed that no significant changes in binding specificity will be observed if most of lysine residues are converted to arginine residues.

TABLE 1
IgG binding parameters of the various proteins prepared

| | | | |
|---|---|---|---|
| Mutation position | | | |
| (residue number) | Kass | Kdis | Kd |
| and amino acid | [M⁻¹s⁻¹] × | [s⁻¹] × | [M] × |

| | | | | | | | | |
|---|---|---|---|---|---|---|---|---|
| Mutant name | 7 | 35 | 49 | 50 | 58 | 10⁻⁵ | 10⁵ | 10¹⁰ |

| | | | | | | | | |
|---|---|---|---|---|---|---|---|---|
| Wild type | Lys | Lys | Lys | Lys | Lys | 1.79 | 6.83 | 3.81 |
| PAA-RRRRG | Arg | Arg | Arg | Arg | Gly | 1.84 | 11.7 | 6.34 |
| (SEQ ID NO: 1) | | | | | | | | |
| PAA-ARRRG | Ala | Arg | Arg | Arg | Gly | 1.97 | 22.4 | 11.3 |
| PAA-ERRRG | Glu | Arg | Arg | Arg | Gly | 2.16 | 16.8 | 7.76 |
| PAA-FRRRG | Phe | Arg | Arg | Arg | Gly | 2.37 | 16.6 | 6.99 |
| PAA-GRRRG | Gly | Arg | Arg | Arg | Gly | 1.67 | 31.0 | 18.5 |
| PAA-HRRRG | His | Arg | Arg | Arg | Gly | 2.02 | 23.2 | 11.5 |
| PAA-IRRRG | Ile | Arg | Arg | Arg | Gly | 2.11 | 13.5 | 6.41 |
| PAA-LRRRG | Leu | Arg | Arg | Arg | Gly | 2.17 | 19.8 | 9.11 |
| PAA-MRRRG | Met | Arg | Arg | Arg | Gly | 2.13 | 12.7 | 5.97 |
| PAA-NRRRG | Asn | Arg | Arg | Arg | Gly | 1.58 | 42.1 | 26.6 |
| PAA-PRRRG | Pro | Arg | Arg | Arg | Gly | 0.86 | 19.1 | 21.6 |
| PAA-QRRRG | Gln | Arg | Arg | Arg | Gly | 1.75 | 25.0 | 14.6 |
| PAA-SRRRG | Ser | Arg | Arg | Arg | Gly | 1.80 | 25.5 | 14.1 |
| PAA-TRRRG | Thr | Arg | Arg | Arg | Gly | 1.04 | 22.2 | 14.4 |
| PAA-VRRRG | Val | Arg | Arg | Arg | Gly | 2.05 | 18.1 | 8.85 |
| PAA-WRRRG | Trp | Arg | Arg | Arg | Gly | 2.73 | 12.1 | 4.43 |
| PAA-RARRG | Arg | Ala | Arg | Arg | Gly | 1.75 | 43.2 | 24.7 |
| PAA-RDRRG | Asp | Arg | Arg | Arg | Gly | 1.64 | 60.8 | 37.1 |
| PAA-RFRRG | Arg | Phe | Arg | Arg | Gly | 1.34 | 78.8 | 59.0 |
| PAA-RGRRG | Arg | Gly | Arg | Arg | Gly | 1.96 | 66.1 | 37.7 |
| PAA-RHRRG | Arg | His | Arg | Arg | Gly | 2.23 | 67.1 | 30.1 |
| PAA-RLRRG | Arg | Leu | Arg | Arg | Gly | 1.96 | 65.9 | 33.6 |
| PAA-RMRRG | Arg | Met | Arg | Arg | Gly | 2.82 | 70.8 | 37.0 |
| PAA-RPRRG | Arg | Pro | Arg | Arg | Gly | 4.52 | 71.5 | 15.8 |
| PAA-RQRRG | Arg | Gln | Arg | Arg | Gly | 4.22 | 53.2 | 12.6 |
| PAA-RSRRG | Arg | Ser | Arg | Arg | Gly | 1.95 | 53.3 | 27.3 |
| PAA-RTRRG | Arg | Thr | Arg | Arg | Gly | 1.50 | 58.6 | 39.1 |
| PAA-RVRRG | Arg | Val | Arg | Arg | Gly | 1.68 | 33.2 | 19.8 |
| PAA-RYRRG | Arg | Tyr | Arg | Arg | Gly | 0.074 | 163 | 2180 |
| PAA-RRARG | Arg | Arg | Ala | Arg | Gly | 2.21 | 9.07 | 4.10 |
| PAA-RRDRG | Arg | Arg | Asp | Arg | Gly | 2.42 | 16.1 | 6.64 |
| PAA-RRHRG | Arg | Arg | His | Arg | Gly | 2.47 | 20.9 | 8.48 |
| PAA-RRMRG | Arg | Arg | Met | Arg | Gly | 2.54 | 30.7 | 12.1 |
| PAA-RRSRG | Arg | Arg | Ser | Arg | Gly | 2.63 | 20.1 | 7.63 |
| PAA-RRTRG | Arg | Arg | Thr | Arg | Gly | 2.40 | 14.2 | 5.91 |
| PAA-RRVRG | Arg | Arg | Val | Arg | Gly | 2.20 | 11.3 | 5.14 |
| PAA-RRWRG | Arg | Arg | Trp | Arg | Gly | 2.50 | 15.5 | 6.21 |
| PAA-RRYRG | Arg | Arg | Tyr | Arg | Gly | 2.38 | 12.5 | 5.25 |
| PAA-RRRAG | Arg | Arg | Arg | Ala | Gly | 2.27 | 18.4 | 8.09 |
| PAA-RRRQG | Arg | Arg | Arg | Gln | Gly | 2.22 | 17.5 | 7.88 |

The thus obtained proteins were immobilized using Amino-Cellulofine (purchased from Seikagaku Corporation) as primary amine carriers. Measurement of the human IgG binding capacity exerted by the thus obtained immobilization carriers is as described in Example 4.

### Example 2

### Conversion to a Sequence Comprising Neither a Cysteine Residue Nor a Lysine Residue and to a Sequence for Immobilization Based on a Sequence Derived from G1 Domain of Streptococcus-Derived Protein G

The sequence derived from G1 domain of Streptococcus-derived protein G is represented by SEQ ID NO: 8.

The above Example demonstrated that original functions were retained even when all lysine residues had been substituted with arginine residues. Accordingly, based on the amino acid sequence represented by SEQ ID NO: 8, the following amino acid sequence (SEQ ID NO: 12) represented by:

Met-Ala-Tyr-Arg-Leu-Ile-Leu-Asn-Gly-Arg-Thr
Leu-Arg-Gly-Glu-Thr-Thr-Thr-Glu-Ala-Val
Asp-Ala-Ala-Thr-Ala-Glu-Arg-Val-Phe-Arg
Gln-Tyr-Ala-Asn-Asp-Asn-Gly-Val-Asp-Gly
Glu-Trp-Thr-Tyr-Asp-Asp-Ala-Thr-Arg-Thr
Phe-Thr-Val-Thr-Glu-Arg-Pro-Glu-Val-Ile
Asp-Ala-Ser-Glu-Leu-Thr-Pro-Ala-Val-Thr
Gly-Gly-Gly-Gly-Cys-Ala-Asp-Asp-Asp-Asp
Asp-Asp-His-His-His-His-His-His

was designed by substituting lysine residues with arginine residues and adding an initiation codon, a spacer sequence, a cysteine-alanine sequence for immobilization reaction, a polyaspartic acid sequence, and a polyhistidine sequence. The following DNA sequence (SEQ ID NO: 13):

GGATCCTTGACAATATCTTAACTATCTGTTATAATATATTGACCAGGTTA
ACTACTAAGCAGCAAAAGGAGGAACGACTATGGCTTACCGTTTAATCCTT
AATGGTCGTACATTGCGTGGCGAAACAACTACTGAAGCTGTTGATGCTGC
TACTGCAGAACGTGTCTTCCGTCAATACGCTAACGACAACGGTGTTGACG
GTGAATGGACTTACGACGATGCGACTCGTACCTTTACGGTAACTGAACGT
CCTGAGGTTATTGATGCTTCGGAGCTGACTCCTGCTGTTACTGGTGGCGG
TGGCTGCGCTGATGACGATGACGATGACCATCATCACCACCATCATTAAG
AATTC

was designed and synthesized, so that a sequence encoding the amino acid sequence of SEQ ID NO: 12 had appropriate transcription initiation functions, translation initiation functions, and a restriction enzyme sequence for incorporation into a vector.

pPG was prepared by inserting the sequence represented by SEQ ID NO: 13 into the BamH I-EcoR I site of a pUC18 vector.

The protein was separated and purified from the Escherichia coli JM109 strain transformed with pPG according to the above-described method. As a result, the target protein was obtained with a yield of approximately 120 mg/2 L of culture solution. The obtained protein was subjected to amino terminal sequence analysis and mass number analysis, so that the amino terminus was found to be alanine and the mass number of the obtained purified protein was found to be 9,698 daltons, as measured using a mass spectrometer. It was confirmed that the obtained protein had been subjected to amino-terminal processing of methione residue corresponding to the initiation codon, as generally observed when a recombinant protein with a sequence containing methionine-alanine as the amino terminal sequence is expressed by Escherichia coli.

The thus obtained protein was examined using Biacore in terms of the activity to bind to human IgG.

Table 2 shows the results. For reference, the values of the protein A mutant protein are shown for comparison.

As shown in Table 2, strong human IgG binding activity was exerted.

TABLE 2
IgG binding parameters of the protein prepared

| | | | |
|---|---|---|---|
| | Kass | | |
| | [M⁻¹s⁻¹] × | Kdis | Kd |
| Protein | 10⁻⁵ | [s⁻¹] × 10⁵ | [M] × 10¹⁰ |

| | | | |
|---|---|---|---|
| pPG | 4.01 | 15.4 | 3.84 |
| (SEQ ID NO: 2) | | | |
| PAA-RRRRG | 1.84 | 11.7 | 6.34 |
| (SEQ ID NO: 1) | | | |

The thus obtained protein was immobilized using Amino-Cellulofine (purchased from Seikagaku Corporation) as a primary amine carrier. Measurement of the human IgG binding capacity exerted by the thus obtained immobilization carrier is described in Example 4.

### Example 3

### Conversion to a Sequence Containing Neither a Cysteine Residue Nor a Lysine Residue and Conversion to a Sequence for Immobilization Based on a Sequence Derived From B1 Domain of Peptostreptococcus-Derived Protein L

A sequence derived from B1 domain of Peptostreptococcus-derived protein L is the sequence represented by SEQ ID NO: 9.

The above Examples demonstrated that original functions were retained even when all lysine residues had been substituted with arginine residues. Hence, based on the amino acid sequence represented by SEQ ID NO: 9, the amino acid sequence (SEQ ID NO: 14) represented by the following sequence:

Met-Ala-Thr-Ile-Arg-Ala-Asn-Leu-Ile-Tyr-Ala
Asp-Gly-Arg-Thr-Gln-Thr-Ala-Glu-Phe-Arg
Gly-Thr-Phe-Glu-Glu-Ala-Thr-Ala-G1U-Ala
Tyr-Arg-Tyr-Ala-Asp-Leu-Leu-Ala-Arg-G1u
Asn-Gly-Arg-Tyr-Thr-Val-Asp-Val-Ala-Asp
Arg-Gly-Tyr-Thr-Leu-Asn-Ile-Arg-Phe-Ala
Gly-Gly-Gly-Gly-Gly-Cys-Ala-Asp-Asp-Asp
Asp-Asp-Asp-His-His-His-His-His-His

was designed by substituting lysine residues with arginine residues and adding an initiation codon, a spacer sequence, a cysteine-alanine sequence for immobilization reaction, and a polyaspartic acid sequence and a polyhistidine sequence. The following DNA sequence (SEQ ID NO: 15) was designed and synthesized so that a sequence encoding the amino acid sequence of SEQ ID NO: 12 had appropriate transcription initiation functions, translation initiation functions, and a restriction enzyme sequence for incorporation into a vector.

GGATCCTTGACAATATCTTAACTATCTGTTATAATATATTGACCAGGTTA
ACTAACTAAGCAGCAAAAGGAGGAACGACTATGGCTACTATTCGTGCTAA
TCTGATTTATGCTGATGGTCGTACTCAGACTGCTGAGTTTCGTGGTACTT
TTGAGGAGGCTACTGCTGAGGCTTATCGTTATGCTGATCTGCTGGCTCGT
GAGAATGGTCGTTATACTGTTGATGTTGCTGATCGTGGTTATACTCTGAA
TATTCGTTTTGCTGGTGGTGGCGGTGGCTGCGCTGATGACGATGACGATG
ACCATCATCACCACCATCATTAAGAATTC

pPL was prepared by inserting the sequence represented by SEQ ID NO: 15 into the BamH I-EcoR I site of a pUC18 vector.

The protein was separated and purified from the Escherichia coli JM109 strain transformed with pPL according to the above-described method. As a result, the target protein was obtained with a yield of approximately 100 mg/2 L of culture solution. The obtained protein was subjected to amino terminal sequence analysis and mass number analysis, so that the amino terminus was found to be alanine and the mass number of the obtained purified protein was found to be 8,782 daltons, as measured using a mass spectrometer. It was confirmed that the obtained protein had been subjected to amino-terminal processing of methione residue corresponding to the initiation codon, as generally observed when a recombinant protein with a sequence containing methionine-alanine as the amino terminal sequence is expressed by Escherichia coli.

The thus obtained protein was examined using Biacore in terms of the activity to bind to human IgG.

Table 3 shows the results. For reference, the values of the protein A mutant protein are shown for comparison.

As shown in Table 3, strong human IgG binding activity was exerted.

TABLE 3
IgG binding parameters of the protein prepared

| | | | |
|---|---|---|---|
| | Kass | | |
| | [M⁻¹s⁻¹] × | Kdis | Kd |
| Protein | 10⁻⁵ | [s^{-1] × 10}⁵ | [M] × 10¹⁰ |

| | | | |
|---|---|---|---|
| pPL | 1.51 | 31.2 | 20.6 |
| (SEQ ID NO: 3) | | | |
| PAA-RRRRG | 1.84 | 11.7 | 6.34 |
| (SEQ ID NO: 1) | | | |

The thus obtained protein was immobilized using Amino-Cellulofine (purchased from Seikagaku Corporation) as a primary amine carrier. Measurement of the human IgG binding capacity exerted by the thus obtained immobilization carrier is described in Example 4.

### Example 4

The proteins (obtained in the above Examples, approximately 6 mg each) comprising the amino acid sequences represented by SEQ ID NOS: 1, 2, and 3, respectively, were each immobilized on 1 ml of Amino-Cellulofine via cyanocysteine-mediated binding reaction. Through the cyanocysteine-mediated binding reaction, immobilization carriers were prepared. Specifically, the sequences represented by SEQ ID NOS: 4, 5, and 6, respectively, were each immobilized on an immobilization carrier in an orientation-controlled manner, so that the carboxy terminus of each sequence was bound to a primary amino group on Amino-Cellulofine via an amide bond. The capacity of binding to human IgG was measured using the thus prepared immobilization carriers (10 μl each), so that the results shown in Table 4 were obtained. Thus, the exertion of the ability of binding to human IgG was confirmed even when orientation-controlled immobilization had been carried out.

TABLE 4

| Immobilization carrier | Amount of bound human IgG |
|---|---|
| (amino acid sequence of protein) | (mg/ml carrier) |
| SEQ ID NO: 4 (←SEQ ID NO: 1) | 12 |
| SEQ ID NO: 5 (←SEQ ID NO: 2) | 10 |
| SEQ ID NO: 6 (←SEQ ID NO: 3) | 3 |

### Example 5

### Preparation of a Protein Having a Repeating Sequence Containing Neither a Cysteine Residue Nor a Lysine Residue Based on a Sequence Derived from Domain a of Staphylococcus-Derived Protein a and Measurement of its IgG Binding Activity

For introduction of a repeating sequence, the following DNA sequence (SEQ ID NO: 27) was designed and synthesized by duplicating a gene encoding a sequence portion (prepared based on a sequence derived from domain A of protein A) containing neither a cysteine residue nor a lysine residue, so that the DNA sequence contained one Cfr9 I cleavage sequence (CCCGGG) as a new restriction enzyme cleavage sequence and could be inserted into a vector via digestion of the entire sequence with BamH I and EcoR I.

(SEQ ID NO: 27)

GGATCCTTGACAATATCTTAACTATCTGTTATAATATATTGACCAGGTTA
ACTAACTAAGCAGCAAAAGGAGGAACGACTATGTCGGGCGGTGGTGGTGC
TGATAACAATTTCAACCGTGAACAACAAAATGCTTTCTATGAAATCTTGA
ATATGCCTAACTTAAACGAAGAACAACGCAATGGTTTCATCCAAAGCTTA
CGTGATGACCCAAGCCAAAGTGCTAACCTATTGTCAGAAGCTCGTCGTTT
AAATGAATCTCAAGCCCCGGGTGCTGATAACAATTTCAACCGTGAACAAC
AAAATGCTTTCTATGAAATCTTGAATATGCCTAACTTAAACGAAGAACAA
CGCAATGGTTTCATCCAAAGCTTACGTGATGACCCAAGCCAAAGTGCTAA
CCTATTGTCAGAAGCTCGTCGTTTAAATGAATCTCAAGCACCGGGTGGTG
GCGGTGGCTGCGCTGATGACGATGACGATGACCATCATCACCACCATCAT
TAAGAATTC

pAAD was prepared by inserting the sequence represented by SEQ ID NO: 27 into the BamH I-EcoR I site of a pUC18 vector. pAAD was expressed by Escherichia coli. Thus, a protein was expressed, comprising the amino acid sequence represented by the general formula R1-R2-R3-R4-R5 having the sequence of SEQ ID NO: 24 repeated twice therein, wherein the sequence of the R1 portion is represented by P-Q,

### P=Ser-Gly-Gly-Gly-Gly (SEQ ID NO: 23)

Q=(Ala-Asp-Asn-Asn-Phe-Asn-Arg-Glu-Gln-Gln-Asn-Ala-Phe-Tyr-Glu-Ile-Leu-Asn-Met-Pro-Asn-Leu-Asn-Glu-Glu-Gln-Arg-Asn-Gly-Phe-Ile-Gln-Ser-Leu-Arg-Asp-Asp-Pro-Ser-Gln-Ser-Ala-Asn-Leu-Leu-Ser-Glu-Ala-Arg-Arg-Leu-Asn-Glu-Ser-Gln-Ala-Pro-Gly) n (where n denotes an arbitrary integer ranging from 2 to 5 and the sequence shown in parentheses is represented by SEQ ID NO: 24),

### R2=Gly-Gly-Gly-Gly (SEQ ID NO: 16),

### R3=Cys-Ala,

### R4=Asp-Asp-Asp-Asp-Asp-Asp (SEQ ID NO: 21), and

### R5=His-His-His-His-His-His (SEQ ID NO: 22).

This protein is represented by n=2. Subsequently, the protein was separated and purified according to the above-described method. The obtained protein was subjected to amino terminal sequence analysis and mass number analysis, so that the amino terminus was found to be serine and the mass number of the obtained purified protein was found to be 15,545 daltons, as measured using a mass spectrometer. It was confirmed that the obtained protein had been subjected to amino-terminal processing of methione residue corresponding to the initiation codon, as generally observed when a recombinant protein with a sequence containing methionine-serine as the amino terminal sequence is expressed by Escherichia coli.

Furthermore, for introduction of a repeating sequence, for which “n” is 3 or a number greater than 3, the following DNA sequence (SEQ ID NO: 28) having a Cfr9 I cleavage sequence (CCCGGG) on its both ends was synthesized.

(SEQ ID NO: 28)

CCCGGGTGCTGATAACAATTTCAACCGTGAACAACAAAATGCTTTCTATG
AAATCTTGAATATGCCTAACTTAAACGAAGAACAACGCAATGGTTTCATC
CAAAGCTTACGTGATGACCCAAGCCAAAGTGCTAACCTATTGTCAGAAGC
TCGTCGTTTAAATGAATCTCAAGCCCCGGG

After digestion with Cfr9 I, the sequence was mixed with pAAD digested with Cfr9 I and then bound with T4DNA ligase. Thus, a recombinant plasmid was prepared, in which one or a plurality of the DNA sequence of SEQ ID NO: 28 that had been digested with Cfr9 I had been bound. The plasmid was digested with BamH I and EcoR I and then subjected to separation by agarose electrophoresis, so that DNA fragments with varied sizes of approximately 0.68 kilobase pairs, approximately 0.86 kilobase pairs, approximately 1.05 kilobase pairs, and larger sizes could be obtained. Each of these DNA fragments was separated from the gel and then introduced into the BamH I-EcoR I site of a pUC18 vector, so that a recombinant plasmid was separated. In the plasmids into which the DNA fragments with an approximately 0.68 kilobase pairs, an approximately 0.86 kilobase pairs, and an approximately 1.05 kilobase pairs had been introduced, respectively, (referred to as pAA3T, pAA4Q, and pAA5P, respectively), one, two, and three portions corresponding to SEQ ID NO: 28 had been bound, respectively. As a result, it was revealed that in the above Q sequence, amino acid sequences corresponding to n=3, 4, and 5, respectively, were encoded. In addition, it was demonstrated that the Escherichia coli JM109 strains transformed with the recombinant plasmids pAA3T, pAA4Q, and pAA5P expressed and accumulated an approximately 22-kilodalton protein, an approximately 29-kilodalton protein, and an approximately 36-kilodalton protein in large amounts.

When the nucleotide sequence of the BamH I-EcoR I site of pAA3T was examined, the sequence was found to be the following DNA sequence.

(SEQ ID NO: 29)

GGATCCTTGACAATATCTTAACTATCTGTTATAATATATTGACCAGGTTA
ACTAACTAAGCAGCAAAAGGAGGAACGACTATGTCGGGCGGTGGTGGTGC
TGATAACAATTTCAACCGTGAACAACAAAATGCTTTCTATGAAATCTTGA
ATATGCCTAACTTAAACGAAGAACAACGCAATGGTTTCATCCAAAGCTTA
CGTGATGACCCAAGCCAAAGTGCTAACCTATTGTCAGAAGCTCGTCGTTT
AAATGAATCTCAAGCCCCGGGTGCTGATAACAATTTCAACCGTGAACAAC
AAAATGCTTTCTATGAAATCTTGAATATGCCTAACTTAAACGAAGAACAA
CGCAATGGTTTCATCCAAAGCTTACGTGATGACCCAAGCCAAAGTGCTAA
CCTATTGTCAGAAGCTCGTCGTTTAAATGAATCTCAAGCCCCGGGTGCTG
ATAACAATTTCAACCGTGAACAACAAAATGCTTTCTATGAAATCTTGAAT
ATGCCTAACTTAAACGAAGAACAACGCAATGGTTTCATCCAAAGCTTACG
TGATGACCCAAGCCAAAGTGCTAACCTATTGTCAGAAGCTCGTCGTTTAA
ATGAATCTCAAGCACCGGGTGGTGGCGGTGGCTGCGCTGATGACGATGAC
GATGACCATCATCACCACCATCATTAAGAATTC

The protein was separated and purified according to the above-described method using Escherichia coli JM109 strain transformed with pAA3T. The obtained protein was subjected to amino terminal sequence analysis and mass number analysis, so that the amino terminus was found to be serine and the mass number of the obtained purified protein was found to be 22,193 daltons, as measured using a mass spectrometer. It was confirmed that the obtained protein had been subjected to amino-terminal processing of methione residue corresponding to the initiation codon, as generally observed when a recombinant protein with a sequence containing methionine-serine as the amino terminal sequence is expressed by Escherichia coli.

The thus obtained protein was examined using Biacore in terms of the activity to bind to human IgG.

Table 5 shows the results. For reference, the values of the mutant protein represented by n=1 are shown for comparison. It was revealed that the value of Kd (the force for binding to IgG) decreased and thus the binding force increased, because of the repeating sequence (Table 5).

TABLE 5

| Plasmid | Number of | kass[M⁻¹s⁻¹] × | koff[s⁻¹] × | Kd[M] × |
|---|---|---|---|---|
| name | repeating unit | 10⁻⁵ | 10⁵ | 10¹⁰ |
| pPAA-RRRRG | n = 1 | 1.84 | 11.7 | 6.34 |
| pAAD | n = 2 | 5.75 | 18.3 | 3.18 |
| pAA3T | n = 3 | 7.86 | 13.3 | 1.69 |

### Example 6

### Preparation of Proteins Having a Repeating Sequence Containing Neither a Cysteine Residue Nor a Lysine Residue Based on a Sequence Derived from G1 Domain of Streptococcus-Derived Protein G and Measurement of its IgG Binding Activity

For introduction of a repeating sequence, the following DNA sequence (SEQ ID NO: 30) was designed and synthesized by duplicating a gene encoding a sequence portion (prepared based on a sequence derived from G1 domain of protein G) containing neither a cysteine residue nor a lysine residue, so that the DNA sequence contained one Cfr9 I cleavage sequence (CCCGGG) as a new restriction enzyme cleavage sequence and could be inserted into a vector via digestion of the entire sequence with BamH I and EcoR I.

(SEQ ID NO: 30)

GGATCCTTGACAATATCTTAACTATCTGTTATAATATATTGACCAGGTTA
ACTAACTAAGCAGCAAAAGGAGGAACGACTATGGCTTACCGTTTAATCCT
TAATGGTCGTACATTGCGTGGCGAAACAACTACTGAAGCTGTTGATGCTG
CTACTGCAGAACGTGTCTTCCGTCAATACGCTAACGACAACGGTGTTGAC
GGTGAATGGACTTACGACGATGCGACTCGTACCTTTACGGTAACTGAACG
TCCTGAGGTTATTGATGCTTCGGAGCTGACTCCTGCTGTTACTCCCGGGG
CTTACCGTTTAATCCTTAATGGTCGTACATTGCGTGGCGAAACAACTACT
GAAGCTGTTGATGCTGCTACTGCAGAACGTGTCTTCCGTCAATACGCTAA
CGACAACGGTGTTGACGGTGAATGGACTTACGACGATGCGACTCGTACCT
TTACGGTAACTGAACGTCCTGAGGTTATTGATGCTTCGGAGCTGACTCCT
GCTGTTACTGGTGGCGGTGGCTGCGCTGATGACGATGACGATGACCATCA
TCACCACCATCATTAAGAATTC

pGGD was prepared by inserting the sequence represented by SEQ ID NO: 30 into the BamH I-EcoR I site of a pUC18 vector. pGGD was expressed by Escherichia coli. Thus, a protein was expressed, comprising the amino acid sequence represented by the general formula R1-R2-R3-R4-R5 having the sequence of SEQ ID NO: 25 repeated twice therein, wherein the sequence of the R1 portion is represented by P-Q,
P=absent,
Q=(Ala-Tyr-Arg-Leu-Ile-Leu-Asn-Gly-Arg-Thr-Leu-Arg-Gly-Glu-Thr-Thr-Thr-Glu-Ala-Val-Asp-Ala-Ala-Thr-Ala-Glu-Arg-Val-Phe-Arg-Gln-Tyr-Ala-Asn-Asp-Asn-Gly-Val-Asp-Gly-Glu-Trp-Thr-Tyr-Asp-Asp-Ala-Thr-Arg-Thr-Phe-Thr-Val-Thr-Glu-Arg-Pro-Glu-Val-Ile-Asp-Ala-Ser-Glu-Leu-Thr-Pro-Ala-Val-Thr-Pro-Gly) n (where n denotes an arbitrary integer ranging from 2 to 5 and the sequence shown in parentheses is represented by SEQ ID NO: 25),

### R2=Gly-Gly-Gly-Gly (SEQ ID NO: 16),

### R3=Cys-Ala,

### R4=Asp-Asp-Asp-Asp-Asp-Asp (SEQ ID NO: 21), and

### R5=His-His-His-His-His-His (SEQ ID NO: 22).

This protein is represented by n=2. Subsequently, the protein was separated and purified according to the above-described method. The obtained protein was subjected to amino terminal sequence analysis and mass number analysis, so that the amino terminus was found to be alanine and the mass number of the obtained purified protein was found to be 17,616 daltons, as measured using a mass spectrometer. It was confirmed that the obtained protein had been subjected to amino-terminal processing of methione residue corresponding to the initiation codon, as generally observed when a recombinant protein with a sequence containing methionine-alanine as the amino terminal sequence is expressed by Escherichia coli.

Furthermore, for introduction of a repeating sequence, for which “n” is 3 or a number greater than 3, the following DNA sequence (SEQ ID NO: 31) having a Cfr9 I cleavage sequence (CCCGGG) on its both ends was synthesized.

(SEQ ID NO: 31)

CCCGGGGCTTACCGTTTAATCCTTAATGGTCGTACATTGCGTGGCGAAAC
AACTACTGAAGCTGTTGATGCTGCTACTGCAGAACGTGTCTTCCGTCAAT
ACGCTAACGACAACGGTGTTGACGGTGAATGGACTTACGACGATGCGACT
CGTACCTTTACGGTAACTGAACGTCCTGAGGTTATTGATGCTTCGGAGCT
GACTCCTGCTGTTACTCCCGGG

After digestion with Cfr9 I, the sequence was mixed with pAAD digested with Cfr9 I and then bound with T4DNA ligase. Thus, a recombinant plasmid was prepared, in which one or a plurality of the DNA sequence of SEQ ID NO: 28 digested with Cfr9 I had been bound. The plasmid was digested with BamH I and EcoR I and then subjected to separation by agarose electrophoresis, so that DNA fragments with varied sizes of approximately 0.79 kilobase pairs, approximately 1.0 kilobase pairs, approximately 1.2 kilobase pairs, and larger sizes could be obtained. Each of these DNA fragments was separated from the gel and then introduced into the BamH I-EcoR I site of a pUC18 vector, so that a recombinant plasmid was separated. In the plasmids, into which approximately 0.79-kilobase-pair, approximately 1.0-kilobase-pair, and approximately 1.2-kilobase-pair DNA fragments had been introduced, respectively, (referred to as pGG3T, pGG4Q, and pGG5P, respectively), one, two, and three portions corresponding to SEQ ID NO: 31 had been bound, respectively. As a result, it was revealed that in the above Q sequence, amino acid sequences corresponding to n=3, 4, and 5, respectively, were encoded. In addition, it was demonstrated that the Escherichia coli JM109 strains transformed with the recombinant plasmids pGG3T, pGG4Q, and pGG5P expressed and accumulated an approximately 25-kilodalton protein, an approximately 33-kilodalton protein, and an approximately 41-kilodalton protein in large amounts.

When the nucleotide sequence of the BamH I-EcoR I site of pGG3T was examined, the sequence was found to be the following DNA sequence.

(SEQ ID NO: 32)

GGATCCTTGACAATATCTTAACTATCTGTTATAATATATTGACCAGGTTA
ACTAACTAAGCAGCAAAAGGAGGAACGACTATGGCTTACCGTTTAATCCT
TAATGGTCGTACATTGCGTGGCGAAACAACTACTGAAGCTGTTGATGCTG
CTACTGCAGAACGTGTCTTCCGTCAATACGCTAACGACAACGGTGTTGAC
GGTGAATGGACTTACGACGATGCGACTCGTACCTTTACGGTAACTGAACG
TCCTGAGGTTATTGATGCTTCGGAGCTGACTCCTGCTGTTACTCCCGGGG
CTTACCGTTTAATCCTTAATGGTCGTACATTGCGTGGCGAAACAACTACT
GAAGCTGTTGATGCTGCTACTGCAGAACGTGTCTTCCGTCAATACGCTAA
CGACAACGGTGTTGACGGTGAATGGACTTACGACGATGCGACTCGTACCT
TTACGGTAACTGAACGTCCTGAGGTTATTGATGCTTCGGAGCTGACTCCT
GCTGTTACTCCCGGGGCTTACCGTTTAATCCTTAATGGTCGTACATTGCG
TGGCGAAACAACTACTGAAGCTGTTGATGCTGCTACTGCAGAACGTGTCT
TCCGTCAATACGCTAACGACAACGGTGTTGACGGTGAATGGACTTACGAC
GATGCGACTCGTACCTTTACGGTAACTGAACGTCCTGAGGTTATTGATGC
TTCGGAGCTGACTCCTGCTGTTACTGGTGGCGGTGGCTGCGCTGATGACG
ATGACGATGACCATCATCACCACCATCATTAAGAATTC

The protein was separated and purified according to the above-described method using Escherichia coli JM109 strain transformed with pGG3T. The obtained protein was subjected to amino terminal sequence analysis and mass number analysis, so that the amino terminus was found to be alanine and the mass number of the obtained purified protein was found to be 25,534 daltons, as measured using a mass spectrometer. It was confirmed that the obtained protein had been subjected to amino-terminal processing of methione residue corresponding to the initiation codon, as generally observed when a recombinant protein with a sequence containing methionine-alanine as the amino terminal sequence is expressed by Escherichia coli.

The thus obtained protein was examined using Biacore in terms of the activity to bind to human IgG.

Table 6 shows the results. For reference, the values of the mutant protein represented by n=1 are shown for comparison. It was revealed that the value of Kd (the force for binding to IgG) decreased and thus the binding force increased because of the repeating sequence (Table 6).

TABLE 6

| Plasmid | Number of | kass[M⁻¹s⁻¹] × | koff[s⁻¹] × | Kd[M] × |
|---|---|---|---|---|
| name | repeating unit | 10⁻⁵ | 10⁵ | 10¹⁰ |
| pPG | n = 1 | 4.01 | 15.4 | 3.84 |
| pGGD | n = 2 | 8.64 | 10.0 | 1.15 |
| pGG3T | n = 3 | 11.2 | 7.63 | 0.68 |

### Example 7

### Preparation of Proteins Having a Repeating Sequence Containing Neither a Cysteine Residue Nor a Lysine Residue Based on a Sequence Derived from B1 Domain of Peptostreptococcus-Derived Protein L and Measurement of its IgG Binding Activity

For introduction of a repeating sequence, the following DNA sequence (SEQ ID NO: 33) was designed and synthesized by duplicating a gene encoding a sequence portion (prepared based on a sequence derived from domain B1 of protein L) containing neither a cysteine residue nor a lysine residue, so that the DNA sequence contained one Cfr9 I cleavage sequence (CCCGGG) as a new restriction enzyme cleavage sequence and could be inserted into a vector via digestion of the entire sequence with BamH I and EcoR I.

(SEQ ID NO: 33)

GGATCCTTGACAATATCTTAACTATCTGTTATAATATATTGACCAGGTTA
ACTAACTAAGCAGCAAAAGGAGGAACGACTATGGCTACTATTCGTGCTAA
TCTGATTTATGCTGATGGTCGTACTCAGACTGCTGAGTTTCGTGGTACTT
TTGAGGAGGCTACTGCTGAGGCTTATCGTTATGCTGATCTGCTGCCTCGT
GAGAATGGTCGTTATACTGTTGATGTTGCTGATCGTGGTTATACTCTGAA
TATTCGTTTTGCTCCCGGGGCTACTATTCGTGCTAATCTGATTTATGCTG
ATGGTCGTACTCAGACTGCTGAGTTTCGTGGTACTTTTGAGGAGGCTACT
GCTGAGGCTTATCGTTATGCTGATCTGCTGCCTCGTGAGAATGGTCGTTA
TACTGTTGATGTTGCTGATCGTGGTTATACTCTGAATATTCGTTTTGCTG
GTGGTGGCGGTGGCTGCGCTGATGACGATGACGATGACCATCATCACCAC
CATCATTAAGAATTC

pLLD was prepared by inserting the sequence represented by SEQ ID NO: 33 into the BamH I-EcoR I site of a pUC18 vector. pLLD was expressed by Escherichia coli. Thus, a protein was expressed, comprising the amino acid sequence represented by the general formula R1-R2-R3-R4-R5 having the sequence of SEQ ID NO: 26 repeated twice therein, wherein the sequence of the R1 portion is represented by P-Q,

P=absent,
Q=(Ala-Thr-Ile-Arg-Ala-Asn-Leu-Ile-Tyr-Ala Asp-Gly-Arg-Thr-Gln-Thr-Ala-Glu-Phe-Arg Gly-Thr-Phe-Glu-Glu-Ala-Thr-Ala-Glu-Ala Tyr-Arg-Tyr-Ala-Asp-Leu-Leu-Ala-Arg-Glu Asn-Gly-Arg-Tyr-Thr-Val-Asp-Val-Ala-Asp Arg-Gly-Tyr-Thr-Leu-Asn-Ile-Arg-Phe-Ala Pro-Gly-) n (where n denotes an arbitrary integer ranging from 2 to 5 and the sequence shown in parentheses is represented by SEQ ID NO: 26),

### R2=Gly-Gly-Gly-Gly (SEQ ID NO: 16),

### R3=Cys-Ala,

### R4=Asp-Asp-Asp-Asp-Asp-Asp (SEQ ID NO: 21), and

### R5=His-His-His-His-His-His (SEQ ID NO: 22).

This protein is represented by n=2. Subsequently, the protein was separated and purified according to the above-described method. The obtained protein was subjected to amino terminal sequence analysis and mass number analysis, so that the amino terminus was found to be alanine and the mass number of the obtained purified protein was found to be 15,779 daltons, as measured using a mass spectrometer. It was confirmed that the obtained protein had been subjected to amino-terminal processing of methione residue corresponding to the initiation codon, as generally observed when a recombinant protein with a sequence containing methionine-alanine as the amino terminal sequence is expressed by Escherichia coli.

Furthermore, for introduction of a repeating sequence, for which “n” is 3 or a number greater than 3, the following DNA sequence (SEQ ID NO: 34) having a Cfr9 I cleavage sequence (CCCGGG) on its both ends was synthesized.

(SEQ ID NO: 34)

CCCGGGGCTACTATTCGTGCTAATCTGATTTATGCTGATGGTCGTACTCA
GACTGCTGAGTTTCGTGGTACTTTTGAGGAGGCTACTGCTGAGGCTTATC
GTTATGCTGATCTGCTGCCTCGTGAGAATGGTCGTTATACTGTTGATGTT
GCTGATCGTGGTTATACTCTGAATATTCGTTTTGCTCCCGGGG

After digestion with Cfr9 I, the sequence was mixed with pAAD digested with Cfr9 I and then bound with T4DNA ligase. Thus, a recombinant plasmid was prepared, in which one or a plurality of the DNA sequence of SEQ ID NO: 34 digested with Cfr9 I had been bound. The plasmid was digested with BamH I and EcoR I and then subjected to separation by agarose electrophoresis, so that DNA fragments with varied sizes of approximately 0.70 kilobase pairs, approximately 0.89 kilobase pairs, approximately 1.1 kilobase pairs, and larger sizes could be obtained. Each of these DNA fragments was separated from the gel and then introduced into the BamH I-EcoR I site of a pUC18 vector, so that a recombinant plasmid was separated. In the plasmids, into which approximately 0.70-kilobase-pair, approximately 0.89-kilobase-pair, and approximately 1.1-kilobase-pair DNA fragments had been introduced, respectively, (referred to as pLL3T, pLL4Q, and pLL5P, respectively), one, two, and three portions corresponding to SEQ ID NO: 34 had been bound, respectively. As a result, it was revealed that in the above Q sequence, amino acid sequences corresponding to n=3, 4, and 5, respectively, were encoded. In addition, it was demonstrated that the Escherichia coli JM109 strains transformed with the recombinant plasmids pLL3T, pLL4Q, and pLL5P expressed and accumulated an approximately 23-kilodalton protein, an approximately 30-kilodalton protein, and an approximately 37-kilodalton protein in large amounts.

When the nucleotide sequence of the BamH I-EcoR I site of pLL3T was examined, the sequence was found to be the following DNA sequence.

(SEQ ID NO: 35)

GGATCCTTGACAATATCTTAACTATCTGTTATAATATATTGACCAGGTTA
ACTAACTAAGCAGCAAAAGGAGGAACGACTATGGCTACTATTCGTGCTAA
TCTGATTTATGCTGATGGTCGTACTCAGACTGCTGAGTTTCGTGGTACTT
TTGAGGAGGCTACTGCTGAGGCTTATCGTTATGCTGATCTGCTGCCTCGT
GAGAATGGTCGTTATACTGTTGATGTTGCTGATCGTGGTTATACTCTGAA
TATTCGTTTTGCTCCCGGGGCTACTATTCGTGCTAATCTGATTTATGCTG
ATGGTCGTACTCAGACTGCTGAGTTTCGTGGTACTTTTGAGGAGGCTACT
GCTGAGGCTTATCGTTATGCTGATCTGCTGCCTCGTGAGAATGGTCGTTA
TACTGTTGATGTTGCTGATCGTGGTTATACTCTGAATATTCGTTTTGCTC
CCGGGGCTACTATTCGTGCTAATCTGATTTATGCTGATGGTCGTACTCAG
ACTGCTGAGTTTCGTGGTACTTTTGAGGAGGCTACTGCTGAGGCTTATCG
TTATGCTGATCTGCTGCCTCGTGAGAATGGTCGTTATACTGTTGATGTTG
CTGATCGTGGTTATACTCTGAATATTCGTTTTGCTGGTGGTGGCGGTGGC
TGCGCTGATGACGATGACGATGACCATCATCACCACCATCATTAAGAATT
C

The protein was separated and purified according to the above-described method using Escherichia coli JM109 strain transformed with pLL3T. The obtained protein was subjected to amino terminal sequence analysis and mass number analysis, so that the amino terminus was found to be alanine and the mass number of the obtained purified protein was found to be 22,751 daltons, as measured using a mass spectrometer. It was confirmed that the obtained protein had been subjected to amino-terminal processing of methione residue corresponding to the initiation codon as generally observed when a recombinant protein with a sequence containing methionine-alanine as the amino terminal sequence is expressed by Escherichia coli.

The thus obtained protein was examined using Biacore in terms of the activity to bind to human IgG.

Table 7 shows the results. For reference, the values of the mutant protein represented by n=1 are shown for comparison. It was revealed that the value of Kd (the force for binding to IgG) decreased and thus the binding force increased, because of the repeating sequence (Table 7).

TABLE 7

| Plasmid | Number of | kass[M⁻¹s⁻¹] × | koff[s⁻¹] × | Kd[M] × |
|---|---|---|---|---|
| name | repeating unit | 10⁻⁵ | 10⁵ | 10¹⁰ |
| pPL | n = 1 | 1.51 | 31.2 | 20.6 |
| pLLD | n = 2 | 2.46 | 26.4 | 13.4 |
| pLL3T | n = 3 | 3.01 | 23.7 | 7.88 |

### INDUSTRIAL APPLICABILITY

With the use of the sequence of the present invention represented by the general formula R1-R2-R3-R4-R5, a subject protein to be immobilized can be efficiently immobilized on an immobilization carrier in an orientation-controlled manner. The product can be used as a protein immobilization carrier for diagnosis, which is used in the medical field such as diagnosis of diseases, an immobilization enzyme, and the like.

### Sequence Listing Free Text

SEQ ID NOS: 1 to 6, 10 to 23, and 27 to 35: Synthesis

1. A protein to be used for immobilizing a portion of the protein represented by R1-R2 on an immobilization carrier having a primary amino group as a functional group only through the carboxy terminus of the portion, comprising an amino acid sequence represented by the general formula R1-R2-R3-R4-R5, wherein:
the sequences are oriented from the amino terminal side to the carboxy terminal side,
the sequence of the R1 portion is the sequence of a subject protein to be immobilized and contains neither a lysine residue nor a cysteine residue;
the sequence of the R2 portion may be absent, but when the sequence of the R2 portion is present, the sequence of the R2 portion is a spacer sequence composed of amino acid residues other than lysine and cysteine residues;
the sequence of the R3 portion is composed of two residues of amino acid represented by cysteine-X (where X denotes an amino acid residue other than lysine or cysteine);
the sequence of the R4 portion may be absent, but when the sequence of the R4 portion is present, the sequence of the R4 portion contains neither a lysine residue nor a cysteine residue, but contains an acidic amino acid residue capable of acidifying the isoelectric point of the entire protein comprising the amino acid sequence represented by the general formula R1-R2-R3-R4-R5; and
the sequence of an R5 portion is an affinity tag sequence for protein purification.

2. The protein according to claim 1, wherein, in the amino acid sequence of the general formula R1-R2-R3-R4-R5, the sequence of the R1 portion is:
the amino acid sequence of a naturally derived protein; or
the amino acid sequence of a protein that comprises an amino acid sequence altered to contain neither a lysine residue nor a cysteine residue and has functions equivalent to those of the naturally derived protein, in which the altered amino acid sequence is obtained by substituting all lysine and cysteine residues in the amino acid sequence of the naturally derived protein with amino acid residues other than lysine and cysteine residues.

3. The protein according to claim 1, wherein, in the amino acid sequence of the general formula R1-R2-R3-R4-R5, the sequence of the R2 portion comprises 1 to 10 glycines.

4. The protein according to claim 1, wherein, in the amino acid sequence of the general formula R1-R2-R3-R4-R5, the sequence of the R4 portion comprises 1 to 10 amino acid residues of aspartic acid and/or glutamic acid.

5. The protein according to claim 1, wherein, in the amino acid sequence of the general formula R1-R2-R3-R4-R5, the sequence of the R5 portion is an amino acid sequence comprising 4 or more histidine residues.

6. The protein according to any one of claims 1 to 5, wherein, in the amino acid sequence of the general formula R1-R2-R3-R4-R5, the sequence of the R1 portion has a function of interacting specifically with an antibody molecule.

7. The protein according to claim 1, comprising the following amino acid sequence (SEQ ID NO: 1):
Ala-Asp-Asn-Asn-Phe-Asn-Arg-Glu-Gln-Gln
Asn-Ala-Phe-Tyr-Glu-Ile-Leu-Asn-Met-Pro
Asn-Leu-Asn-Glu-Glu-Gln-Arg-Asn-Gly-Phe
Ile-Gln-Ser-Leu-Arg-Asp-Asp-Pro-Ser-Gln
Ser-Ala-Asn-Leu-Leu-Ser-Glu-Ala-Arg-Arg
Leu-Asn-Glu-Ser-Gln-Ala-Pro-Gly-Gly-Gly
Gly-Gly-Cys-Ala-Asp-Asp-Asp-Asp-Asp-Asp
His-His-His-His-His-His

8. The protein according to claim 1, comprising the following sequence (SEQ ID NO: 2):
Ala-Tyr-Arg-Leu-Ile-Leu-Asn-Gly-Arg-Thr
Leu-Arg-Gly-Glu-Thr-Thr-Thr-Glu-Ala-Val
Asp-Ala-Ala-Thr-Ala-Glu-Arg-Val-Phe-Arg
Gln-Tyr-Ala-Asn-Asp-Asn-Gly-Val-Asp-Gly
Glu-Trp-Thr-Tyr-Asp-Asp-Ala-Thr-Arg-Thr
Phe-Thr-Val-Thr-Glu-Arg-Pro-Glu-Val-Ile
Asp-Ala-Ser-Glu-Leu-Thr-Pro-Ala-Val-Thr
Gly-Gly-Gly-Gly-Cys-Ala-Asp-Asp-Asp-Asp
Asp-Asp-His-His-His-His-His-His

9. The protein according to claim 1, comprising the following sequence (SEQ ID NO: 3):
Ala-Thr-Ile-Arg-Ala-Asn-Leu-Ile-Tyr-Ala
Asp-Gly-Arg-Thr-Gln-Thr-Ala-Glu-Phe-Arg
Gly-Thr-Phe-Glu-Glu-Ala-Thr-Ala-Glu-Ala
Tyr-Arg-Tyr-Ala-Asp-Leu-Leu-Ala-Arg-Glu
Asn-Gly-Arg-Tyr-Thr-Val-Asp-Val-Ala-Asp
Arg-Gly-Tyr-Thr-Leu-Asn-Ile-Arg-Phe-Ala
Gly-Gly-Gly-Gly-Gly-Cys-Ala-Asp-Asp-Asp
Asp-Asp-Asp-His-His-His-His-His-His

10. The protein according to claim 1, wherein, in the amino acid sequence represented by the general formula R1-R2-R3-R4-R5, the sequence of the R1 portion is represented by P-Q, the sequence of the P portion may be present or absent and is a sequence comprising (Ser or Ala)-(Gly)n (where n denotes an arbitrary integer ranging from 1 to 10) when present, and the sequence of the Q portion is the sequence of a protein having a repeating unit in which a sequence unit containing neither a lysine residue nor a cysteine residue is repeated.

11. The protein according to claim 10, wherein, in the amino acid sequence represented by P-Q, the sequence of the repeating unit of the Q portion is the amino acid sequence of a naturally derived protein or the amino acid sequence of a protein that comprises an amino acid sequence altered to contain neither a lysine residue nor a cysteine residue, which is obtained by substituting all lysine and cysteine residues in the amino acid sequence of the naturally derived protein with amino acid residues other than lysine and cysteine residues and has functions equivalent to those of the naturally derived protein.

12. The protein according to claim 10, wherein, in the amino acid sequence represented by the general formula R1-R2-R3-R4-R5, the sequence of the R2 portion comprises 1 to 10 glycines.

13. The protein according to claim 10, wherein, in the amino acid sequence represented by the general formula R1-R2-R3-R4-R5, the sequence of the R4 portion comprises 1 to 10 amino acid residues comprising amino acid residues, aspartic acid and/or glutamic acid.

14. The protein according to claim 10, wherein, in the amino acid sequence represented by the general formula R1-R2-R3-R4-R5, the sequence of the R5 portion is an amino acid sequence comprising 4 or more histidine residues.

15. The protein according to any one of claims 10 to 14, wherein, in the amino acid sequence of the general formula R1-R2-R3-R4-R5, the sequence of the repeating unit of the Q portion has a function of interacting specifically with an antibody molecule when the sequence of the R1 portion is represented by P-Q.

16. The protein according to claim 10, wherein, in the amino acid sequence represented by the general formula R1-R2-R3-R4-R5, the sequence of the R1 portion is represented by P-Q,
P=Ser-Gly-Gly-Gly-Gly,
Q=(Ala-Asp-Asn-Asn-Phe-Asn-Arg-Glu-Gln-Gln-Asn-Ala-Phe-Tyr-Glu-Ile-Leu-Asn-Met-Pro-Asn-Leu-Asn-Glu-Glu-Gln-Arg-Asn-Gly-Phe-Ile-Gln-Ser-Leu-Arg-Asp-Asp-Pro-Ser-Gln-Ser-Ala-Asn-Leu-Leu-Ser-Glu-Ala-Arg-Arg-Leu-Asn-Glu-Ser-Gln-Ala-Pro-Gly) n (where n denotes an arbitrary integer ranging from 2 to 5),
R2=Gly-Gly-Gly-Gly,
R3=Cys-Ala,
R4=Asp-Asp-Asp-Asp-Asp-Asp, and
R5=His-His-His-His-His-His.

17. The protein according to claim 10, wherein, in the amino acid sequence represented by the general formula R1-R2-R3-R4-R5, the sequence of the R1 portion is represented by P-Q,
P=absent,
Q=(Ala-Tyr-Arg-Leu-Ile-Leu-Asn-Gly-Arg-Thr-Leu-Arg-Gly-Glu-Thr-Thr-Thr-Glu-Ala-Val-Asp-Ala-Ala-Thr-Ala-Glu-Arg-Val-Phe-Arg-Gln-Tyr-Ala-Asn-Asp-Asn-Gly-Val-Asp-Gly-Glu-Trp-Thr-Tyr-Asp-Asp-Ala-Thr-Arg-Thr-Phe-Thr-Val-Thr-Glu-Arg-Pro-Glu-Val-Ile-Asp-Ala-Ser-Glu-Leu-Thr-Pro-Ala-Val-Thr-Pro-Gly) n (where n denotes an arbitrary integer ranging from 2 to 5),
R2=Gly-Gly-Gly-Gly,
R3=Cys-Ala,
R4=Asp-Asp-Asp-Asp-Asp-Asp, and
R5=His-His-His-His-His-His.

18. The protein according to claim 10, wherein, in the amino acid sequence represented by the general formula R1-R2-R3-R4-R5, the sequence of the R1 portion is represented by P-Q,
P=absent,
Q=(Ala-Thr-Ile-Arg-Ala-Asn-Leu-Ile-Tyr-Ala Asp-Gly-Arg-Thr-Gln-Thr-Ala-Glu-Phe-Arg Gly-Thr-Phe-Glu-Glu-Ala-Thr-Ala-Glu-Ala Tyr-Arg-Tyr-Ala-Asp-Leu-Leu-Ala-Arg-Glu Asn-Gly-Arg-Tyr-Thr-Val-Asp-Val-Ala-Asp Arg-Gly-Tyr-Thr-Leu-Asn-Ile-Arg-Phe-Ala Pro-Gly-) n (where n denotes an arbitrary integer ranging from 2 to 5),
R2=Gly-Gly-Gly-Gly,
R3=Cys-Ala,
R4=Asp-Asp-Asp-Asp-Asp-Asp, and
R5=His-His-His-His-His-His

19. A method for preparing an immobilized protein bound to an immobilization carrier having a primary amino group as a functional group using the protein according to claim 1, wherein a portion represented by R1-R2 of the protein is bound to the carrier only through the carboxy terminus of the portion, comprising:
converting a sulfhydryl group of the sole cysteine residue existing in R3 in the amino acid sequence represented by the general formula R1-R2-R3-R4-R5 to a thiocyano group; and then
causing the resultant to act on an immobilization carrier having a primary amino group as a functional group, so as to bind the carboxy terminus of the R1-R2 amino acid sequence portion existing on the amino terminal side from the cysteine residue in the protein to the immobilization carrier via an amide bond.

20. An immobilized protein, to which a protein comprising the amino acid sequence represented by the general formula R1-R2 (where, R1 and R2 have the same meaning as that of R1 and R2 in the general formula according to claim 1) is bound to an immobilization carrier, wherein
the protein is bound the immobilization carrier having a primary amino group as a functional group via an amide bond only through the carboxy terminus of R1-R2.

21. The immobilized protein according to claim 20 prepared using the protein according to claim 1, wherein:
a portion represented by R1-R2 of the protein is bound to an immobilization carrier having a primary amino group as a functional group only through the carboxy terminus; and
a sulfhydryl group of the sole cysteine residue existing in R3 in the amino acid sequence represented by the general formula R1-R2-R3-R4-R5 is converted to a thiocyano group and then the resultant is caused to act on an immobilization carrier having a primary amino group as a functional group, so as to bind the carboxy terminus of the R1-R2 amino acid sequence portion existing on the amino terminal side from the cysteine residue in the protein to the immobilization carrier via an amide bond.
